SEQUENCE LISTING

SEQ ID NO: 1 - DNA, 2592 bp, Human immunodeficiency virus type 1
  misc_feature: GenBank Accession No. AF443202
atgagagtgaaggggatcaggaggaattatcagcactggtggggatggggcacgatgctccttgggctattaatgatctgtagtgctacagaaaaattgtgggtcacagtctattatggggtacctgtgtggaaagaagcaaccaccactctattttgtgcatcagatgctaaagcatatgatacagaggtacataatgtttgggccacacatgcctgtgtacccacagaccccaacccacaagaagtacaattggtaaatgtgacagaagattttaacatgtggaaaaataacatggtagaacagatgcatgaggatataatcagtttatgggatcaaagcctaaagccatgtgtaaaattaaccccactctgtgttactttaaattgcactgatttgaggaatacaaattgcactgattcgaatacaacttgcactaatttggggaatactactaatgatagtaacaagacaacgatggagggaggagaaatgaaaaactgctctttcaatatcaccacaagcataagagataagatgcagaaagaatatgcacttctttataaacttgatatagtagcaatagataaggataataccagctataggttaataagttgtaatacctcagtcattacacaagcctgtccaaaggtatcctttgagccaattcccatacattattgtgccccggctggttttgcgattctaaagtgtaatgataaaaatttcactggaaaaggaccatgtaaaaatgtcagcacagtacaatgtacacatggaattaggccagtagtatcaactcaactgctgttaaatggcagtctagcagaagaagaggtagtaattagatctgagaatttcactaataatgctaaaaccatcatagtacatctgaatgaatccgtacaaattaattgtacaagaccctacaacaatagaagaacaaggatacatataggaccagggagagcattttatacaacaaaaaatataaaaggaactataagacaagcacattgtaccattagtagcgcaaaatggaatgacactttaagacagatagttagcaaattaaaagaacaatttaagaataaaacaatagtctttaaacaatcctcaggaggggacccagaaattgtaatgcacagttttaattgtggaggggaatttttctactgtaatacatcatcactgtttaatagtacttggaatggtaataatacttggaataatactacagggtcaaatagcaatatcacacttcaatgcaaaataaaacaaattataaacatgtggcaggaagtaggaaaagcaatgtatgcccctcccattgaagggcaaattagatgttcatcaaatattacaggactgctattaacaagagatggtggtaatgatacggatacgaacaacaccgagatcttcagacctggaggaggagatatgagggacaattggagaagtgaattatataaatataaagtagtaacaattgaaccattaggagtagcacccaccaaggcaaaaagaagagtggtgcagagagaaaaaagagcagcgataggagctctgttccttgggttcttaggagcagcaggaagcactatgggcgcagcgtcaatgatgctgacggtacaggccagacaattattgtctggtatagtgcaacagcagaacaatttgctgagggccattgaggcgcaacagcatatgttgcaactcacagtctggggcataaagcaactccaggcaagagtcctggctgtggaaagatacctaagggatcaacagctcctggggatttggggttgctctggaaaactcatttgcaccactactgtgccttggaatgctagttggagtaataaatctcaggaggatatttggaataacatgacctggatgcagtgggaaagagaaattgacaattacacaagcacaatatacgaattacttgaaaaatcgcaaaaccaacaagaaaagaatgaacaagaattattggaattagataaatgggcaagtttgtggaattggtttgacataacaaattggctgtggtatataaaaatattcataatgatagtaggaggcttgataggtttaagaatagtttttgctgtactttctatagtgaatagagttaggcagggatactcaccattgtcgttgcagacccgccccccagttccgaggggacccgacaggcccgaaggaaccgaagaagaaggtggagagagagacagagacacatccggacgattagtggatggattcttagcaattatctgggtcgacctgcggagcctgttactcttcagctaccaccgcttgagagacttactcttgattgcagcgaggattgtggaacttctgggacgcagggggtgggaaatcctcaaatattggtggaatctcctacagtattggagtcaggaactaaagaatagtgccgttagcttgcttaatgccacagctgtagcagtagctgaggggacagatagggttatagaagtattgcaaagagctggtagagctattctccacatacctacaagaataagacagggcttggaaagggctttgctataa

SEQ ID NO: 2 - protein, 863 aa, Human immunodeficiency virus type 1
  misc_feature: GenBank Accession No. AF443202
MRVKGIRRNYQHWWGWGTMLLGLLMICSATEKLWVTVYYGVPVWKEATTTLFCASDAKAYDTEVHNVWATHACVPTDPNPQEVQLVNVTEDFNMWKNNMVEQMHEDIISLWDQSLKPCVKLTPLCVTLNCTDLRNTNCTDSNTTCTNLGNTTNDSNKTTMEGGEMKNCSFNITTSIRDKMQKEYALLYKLDIVAIDKDNTSYRLISCNTSVITQACPKVSFEPIPIHYCAPAGFAILKCNDKNFTGKGPCKNVSTVQCTHGIRPVVSTQLLLNGSLAEEEVVIRSENFTNNAKTIIVHLNESVQINCTRPYNNRRTRIHIGPGRAFYTTKNIKGTIRQAHCTISSAKWNDTLRQIVSKLKEQFKNKTIVFKQSSGGDPEIVMHSFNCGGEFFYCNTSSLFNSTWNGNNTWNNTTGSNSNITLQCKIKQIINMWQEVGKAMYAPPIEGQIRCSSNITGLLLTRDGGNDTDTNNTEIFRPGGGDMRDNWRSELYKYKVVTIEPLGVAPTKAKRRVVQREKRAAIGALFLGFLGAAGSTMGAASMMLTVQARQLLSGIVQQQNNLLRAIEAQQHMLQLTVWGIKQLQARVLAVERYLRDQQLLGIWGCSGKLICTTTVPWNASWSNKSQEDIWNNMTWMQWEREIDNYTSTIYELLEKSQNQQEKNEQELLELDKWASLWNWFDITNWLWYIKIFIMIVGGLIGLRIVFAVLSIVNRVRQGYSPLSLQTRPPVPRGPDRPEGTEEEGGERDRDTSGRLVDGFLAIIWVDLRSLLLFSYHRLRDLLLIAARIVELLGRRGWEILKYWWNLLQYWSQELKNSAVSLLNATAVAVAEGTDRVIEVLQRAGRAILHIPTRIRQGLERALL

SEQ ID NO: 3 - DNA, 2574 bp, Human immunodeficiency virus type 1
  misc_feature: GenBank Accession No. AF075721
atgagagtgaaagggatcaggaggaattatcagcactggtggggatggggcacgatgctccttgggttattaatgatctgtagtgctacagaaaaattgtgggtcacagtctattatggggtacctgtgtggaaagaagcaaccaccactctattttgtgcatcagatgctaaagcatatgatacagaggcacataatgtttgggccacacatgcctgtgtacccacagaccccaacccacaagaagtagaattggtaaatgtgacagaaaattttaacatgtggaaaaataacatggtagaacagatgcatgaggatataatcagtttatgggatcaaagcctaaagccatgtgtgaaattaaccccactctgtgttactttaaattgcactgatttgaggaatactactaataccaatgatagtactgctaataacaatagtaatagcgagggaacgataaagggaggagaaatgaaaaactgctctttcaatatcaccacaagcataggaaataagatgcagaaagaatatgcacttctttataaacttgatatagaaccaatagataatgatagtaccagccataggttgataagttgtaatacctcagtcattacacaagcttgtccaaagatatcctttgagccaattcccatacactattgtgccccggctggttttgcgattctaaagtgtaacgataaaaagttcagcggaaaaggatcatgtaaaaatgtcagcacagtacaatgtacacatggaattaggccagtagtatcaactcaactgctgttaaatggcagtctagcagaagaagaggtagtaattagatctgaggatttcactgataatgctaaaaccatcatagtacatctgaaagaatctgtacaaattaattgtacaagacccaactacaataaaagaaaaaggatacatataggaccagggagagcattttatacaacaaaaaatataaaaggaactataagacaagcacattgtaccattagtagagcaaaatggaatgacactttaagacagatagttagcaagttaaaagaacaatttaagaataaaacgatagtctttaatccatcctcaggaggggacccagaaattgtaatgcacagttttaattgtggaggggaatttttctactgtaatacatcaccactgtttaatagtacttggaatggtaataatacttggaataatactacagggtcaaataacaatatcacacttcaatgcaaagtaaaacaaattataaacatgtggcagaaagtaggaaaagcaatgtatgcccctcccattgaaggacaaattagatgttcatcaaatattacagggctactattaacaagagatggtggtgaggacacggacacgaacgacaccgagatcttcagacctggaggaggagatatgagggacaattggagaagtgaattatataaatataaagtagtaacaattgaaccattaggagtagcacccaccaaggcaaagagaagagtggtgcagagagaaaaaagagcagcgataggagctctgttccttgggttcttaggagcagcaggaagcactatgggcgcagcgtcagtgacgctgacggtacaggccagactattattgtctggtatagtgcaacagcagaacaatttgctgagggccattgaggcgcaacagaatatgttgcaactcacagtctggggcatcaagcagctccaggcaagagtccaggctgtggaaagatacctaaaggatcaacagctcctggggttttggggttgctctggaaaactcatttgcaccactactgtgccttggaatgctagttggagtaataaatccctggatgatatttggaataacatgacctggatgcagtgggaaagagaaattgacaattacacaagcttaatatactcattactagaaaaatcgcaaacccaacaagaaaagaatgaacaagaattattgggattggataaatgggaaagcttgtggaattggtttgacataacaaattggctgtggtatataaaaatattcataatgatagtaggaggcttggtaggtttaagaatagtttttgctgtactttctatagtgaatagagttaggcagggatactcaccattgtcgttgcagacccgccccccagttccgaggggacccgacaggcccgaaggaatcgaagaagaaggtggagagagagacagagacacatccggtcgattagtgcatggattcttagcaattatctgggtcgacctgcggagcctgttcctcctcagctaccaccacttgagagacttactcttgattgcagcgaggattgtggaacttctgggacgcagggggtgggaagtcctcaaatattggtggaatctcctacagtactggagtcaggaactaaagagtagtgctgttagcttgcttaatgccacagctatagcagtagctgaggggacaaatagggttatagaagtactgcaaagagctggtagagctattctccacatacccacaagaataagacagggcttggaaagggctttgctataa

SEQ ID NO: 4 - protein, 857 aa, Human immunodeficiency virus type 1
  misc_feature: GenBank Accession No. AF075721
MRVKGIRRNYQHWWGWGTMLLGLLMICSATEKLWVTVYYGVPVWKEATTTLFCASDAKAYDTEAHNVWATHACVPTDPNPQEVELVNVTENFNMWKNNMVEQMHEDIISLWDQSLKPCVKLTPLCVTLNCTDLRNTTNTNDSTANNNSNSEGTIKGGEMKNCSFNITTSIGNKMQKEYALLYKLDIEPIDNDSTSHRLISCNTSVITQACPKISFEPIPIHYCAPAGFAILKCNDKKFSGKGSCKNVSTVQCTHGIRPVVSTQLLLNGSLAEEEVVIRSEDFTDNAKTIIVHLKESVQINCTRPNYNKRKRIHIGPGRAFYTTKNIKGTIRQAHCTISRAKWNDTLRQIVSKLKEQFKNKTIVFNPSSGGDPEIVMHSFNCGGEFFYCNTSPLFNSTWNGNNTWNNTTGSNNNITLQCKVKQIINMWQKVGKAMYAPPIEGQIRCSSNITGLLLTRDGGEDTDTNDTEIFRPGGGDMRDNWRSELYKYKVVTIEPLGVAPTKAKRRVVQREKRAAIGALFLGFLGAAGSTMGAASVTLTVQARLLLSGIVQQQNNLLRAIEAQQNMLQLTVWGIKQLQARVQAVERYLKDQQLLGFWGCSGKLICTTTVPWNASWSNKSLDDIWNNMTWMQWEREIDNYTSLIYSLLEKSQTQQEKNEQELLGLDKWESLWNWFDITNWLWYIKIFIMIVGGLVGLRIVFAVLSIVNRVRQGYSPLSLQTRPPVPRGPDRPEGIEEEGGERDRDTSGRLVHGFLAIIWVDLRSLFLLSYHHLRDLLLIAARIVELLGRRGWEVLKYWWNLLQYWSQELKSSAVSLLNATAIAVAEGTNRVIEVLQRAGRAILHIPTRIRQGLERALL

(19) United States
(12) Patent Application Publication
(10) Pub. No.: US 2008/0057492 A1
(43) Pub. Date: Mar. 6, 2008

(54) Interacting site for gp41 on gp120 of hiv-1

(76) Inventor: Gerald V. Quinnan Jr, Bethesda, MD (US)

Correspondence Address: MORGAN LEWIS & BOCKIUS LLP, 1111 PENNSYLVANIA AVENUE NW, WASHINGTON, DC, 20004, US

(21) Appl. No.: 10/513,884

(22) PCT Filed: May 12, 2003
(86) PCT No.: PCT/US03/14721

§ 371 Date: May 23, 2005

(30) Foreign Application Priority Data
May 10, 2002 (US) 60379052

(51) Int. Cl. C12Q 1/70; C07K 14/16

(52) U.S. Cl. 435/5; 530/350

(57) ABSTRACT

Mutations in the amino and carboxy terminal halves of HIV-1 gp120 and the carboxy terminus of gp41 are disclosed which contribute to the neutralization resistance and high infectivity phenotypes of HIV-1.

### RELATED APPLICATION DATA

This application claims the benefit of U.S. Provisional Application 60/379,052 (filed May 10, 2002) which is herein incorporated by reference in its entirety.

### ACKNOWLEDGEMENT OF FEDERAL SUPPORT

The present invention arose in part from research funded by a federal grant from the National Institutes of Health (AI 37438).

### FIELD OF THE INVENTION

The invention relates to the field of virology and in particular, human immunodeficiency virus (HIV) and Acquired Immune Deficiency Syndrome (AIDS). The invention specifically relates to HIV-1 envelope protein amino acid sequences that modulate interactions between different regions of the oligomeric protein complex and determine the immunological phenotype of the envelope.

### BACKGROUND

The induction of a broadly protective neutralizing antibody response is a major concern regarding the potential efficacy of vaccines. Efforts to develop a vaccine against HIV-1 have been slow as a result of resistance of virus to neutralization, and difficulties in preparing envelope protein in a stable conformation that expresses conserved neutralization epitopes. This resistance is manifest in comparisons of primary virus isolates to laboratory adapted viruses and antigenic variations among strains (Back et al. (1994) Virology 199, 431-438; Berman et al. (1997)J. Infect. Dis. 176, 384-397; Katzenstein et al. (1990) J. Acquir. Immune Defic. Syndr. 3, 810-816; Laman et al. (1992) J. Virol. 66, 1823-1831).

In some lentivirus infections disease episodes occur intermittently, and may be related to periods of increased viral replication that may follow occurrence of escape mutations (Konno & Yamamoto (1990) Cornell Vet. 60, 393-449). In HIV infection there is evidence of partial immune control of viral replication. Virus is readily detected in plasma early during acute infection, but at lower levels during the pre-AIDS period of chronic infection Montefiori et al. (1996) J. Infect. Dis. 173, 60-67). Despite the partial control of replication, substantial virus replication remains ongoing during the chronic, pre-AIDS period of infection, with the potential for mutant strains to emerge (Ho et al. (1989) N. Engl. J. Med. 321, 1621-1625; Perelson et al. (1996) Science 271, 1582-1586). Neutralization escape mutations may be important during the period of chronic infection, or as an even contributing to late immunological deterioration.

Limited studies have been performed of neutralization escape mutations occurring in vivo, or in vitro in the presence of sera from infected people or animals (McKnight & Clapham (1995) Trends Microbiol. 3, 356-361; Park & Quinnan (1999) J. Virol. 73, 5707-5713). Two groups have reported evidence of V3 region escape mutation occurring early during the course of infection (Sawyer et al. (1990) AIDS Res. Hum. Retroviruses 6, 341-356; Zhang et al. (2002) J. Virol. 76, 644-655). Others have reported mutations at sites distant from neutralization epitopes, in gp41, that mediate a global resistance phenotype affecting neutralization by antibodies against multiple epitopes (Back et al. (1994) Virology 199, 431-438; Matsushita et al. (1988) J. Virol. 62, 2107-2114; McKeating et al. (1992) Virology 191, 732-742; Reitz et al. (1988) Cell 54, 57-63; Wyatt et al. (1993) J. Virol. 67, 4557-4565). It has been considered likely that these mutations contribute to neutralization resistance through effects on conformation of the envelope complex.

The determination of the atomic structure of gp120 and the discovery that chemokine receptors are co-receptor for HIV have substantially advanced understanding of the nature of neutralization epitopes on the envelope complex, and the potential role of these epitopes in cell attachment and entry (Alkhatib et al. (1996) Science 272, 1955-1958; Deng et al. (1996); Feng et al. (1996) Science 272, 872-877; Konno et al. (1970) Cornell Vet. 60, 393-449; Wyatt et al. (1998) Nature 393, 705-711). The neutralization epitopes that are functional on primary envelopes tend to be conformation dependent (Fouts et al. (1977) J. Virol. 71, 2779-2785). Accessibility of some of the epitopes depends on conformational changes that occur after engagement of CD4. These CD4-induced epitopes are generally thought to be epitopes in the co-receptor-binding site. As a result of poor neutralizing antibody responses to experimental vaccines, there has developed interest in defining methods to induce antibodies against epitopes exposed during the conformational changes that follow receptor engagement. Characterization of the mechanisms of neutralization resistance and of target epitopes that may be functional in neutralization of primary isolates may substantially facilitate efforts to immunize effectively against HIV.

### SUMMARY OF THE INVENTION

The invention encompasses a method of identifying a human immuno-deficiency virus type-1 (HIV-1) envelope protein which produces a cross-reactive immune response following administration in a mammal comprising substituting one or more amino acids in the gp41, CD4 or co-receptor binding domain of gp120, or the outer domain of gp120, and identifying one or more amino acid substitutions in the binding domains that produce a cross-reactive immune response following administration in a mammal. These substitutions may be in the C1, C2, C3, C4, C5, V1/V2, V3, V4 and/or V5 domains of gp120 and be located at a position corresponding to an amino acid position in gp120 selected from the group consisting of amino acids 64, 84, 91, 219, 243, 245, 249, 287, 290, 300, 211, 312, 314, 316, 345, 371, 372, 398, 426, 418, 435, 466 and 472. In some embodiments the substitution is selected from the group consisting of D91N, V219I, N243K, T245S and P249S, D287N, D290N, N371K, P372Q, V426I, K435N, E466N and D472N. Furthermore, at least one nucleotide in a nucleic acid encoding the HIV-1 envelope protein is substituted.

The invention also encompasses any HIV-1 envelope protein identified by the method the invention including an isolated HIV-1 envelope protein comprising one or more amino acid substitutions at a position corresponding to an amino acid in gp120 selected from the group consisting of amino acids 64, 84, 91, 219, 243, 245, 249, 287, 290, 300, 311, 312, 314, 316, 345, 371, 372, 398, 418, 426, 435, 466 and 472.

The invention further encompasses an isolated HIV-1 envelope protein comprising at least one first fragment of an antibody neutralization-resistant envelope protein and at least one second fragment from an antibody neutralization-sensitive envelope protein. In some embodiments the first or second fragment is about 50 to about 800 amino acids. In one embodiment the first fragment from the neutralization-resistant envelope protein comprises a fragment from SEQ ID NO: 2 (MN-P). Exemplary fragments include, but are not limited to, amino acids 1 to 123 of SEQ ID NO: 2, 123 to 212 of SEQ ID NO: 2, 212 to 274 of SEQ ID NO: 2, 274 to 367 of SEQ ID NO: 2, 367 to 468 of SEQ ID NO: 2, 468 to 517 of SEQ ID NO: 2, 517 to 611 of SEQ ID NO: 2, 611 to 759 of SEQ ID NO: 2, and 759 to 858 of SEQ ID NO: 2. In another embodiment, the second fragment is from a antibody neutralization-sensitive envelope protein and comprises a fragment from SEQ ID NO: 4 (MN-TCLA). Exemplary fragments include, but are not limited to, amino acids 1 to 123 of SEQ ID NO: 4, 123 to 212 of SEQ ID NO: 4, 212 to 274 of SEQ ID NO: 4, 274 to 367 of SEQ ID NO: 4, 367 to 468 of SEQ ID NO: 4, 468 to 517 of SEQ ID NO: 4, 517 to 611 of SEQ ID NO: 4, 611 to 759 of SEQ ID NO: 4, and 759 to 858 of SEQ ID NO: 4.

In some embodiments, the above-mentioned HIV-1 envelope protein of the invention further comprises one or more amino acid substitutions. These substitutions can be at an amino acid position selected from the group consisting of amino acids 64, 84, 91, 219, 243, 245, 249, 287, 290, 300, 311, 312, 314, 316, 345, 371, 372, 398, 418, 426, 435, 466 and 472. Examples of these substitutions include but are not limited to, one or more amino acid substitutions selected from the group consisting of V64A, Q84E, N243K, T245S, P249S, D287N, D290N, N311Y, Y312N, K314R, K316T, N371K, P372Q, V426I, K435N, E466N and D472N. The invention also encompasses a fusion protein comprising any of the HIV-1 envelope proteins of the invention.

The invention includes a nucleic acid molecule encoding any of the above HIV-1 envelope proteins of the invention. In some embodiments, the nucleic acid molecule is operably linked to one or more expression control elements. Vectors comprising an isolated nucleic acid molecule and host cells containing these vectors (including viral vectors) are also within the scope of the invention. The invention also includes a method for producing the HIV-1 envelope proteins of the invention comprising culturing a host cell transformed with the nucleic acid molecule encoding this protein under conditions in which the polypeptide encoded by said nucleic acid molecule is expressed.

The invention further includes a composition comprising the HIV-1 envelope protein of the invention and a pharmaceutically acceptable carrier. Such compositions include immunogenic and vaccine compositions. Also within the scope of the invention is an attenuated HIV-1 comprising any of the nucleic acid molecule of the invention encoding a HIV-1 envelope protein.

### BRIEF DESCRIPTION OF THE DRAWINGS

FIG. 1. Alignment of amino acid sequences of envelopes of neutralization sensitive (MN-TCLA), highly neutralization resistant (MN-P and MN-P14) and partially neutralization resistant (MN-E6) clones of the HIV-1 MN strain. The bars above the amino-acid sequences indicate the approximate locations of regions of the glycoproteins, as follows, C1, C2, C3, C4=constant regions of gp120; V1/V2, V3, V4, V5=variable regions of gp120; gp41 regions=FP (fusion peptide), LZ (leucine zipper), AH (membrane proximal alpha helix), TM (transmembrane segment), and CT (cytoplasmic tail). Positions of amino acids are numbered as in MN-P, and amino acids differing from consensus are boxed and shaded. Residues numbers appear in the boxes at locations corresponding to restriction enzyme cleavage sites illustrated in FIG. 2.

FIG. 2. The gp41 Leucine Zipper interacts functionally with multiple regions of gp120 and gp41 to determine the neutralization resistance, high infectivity phenotype. (A) Restriction endonuclease cleavage map of the MN-TCLA clone indicating nucleotide positions of cleavage sites for specific enzymes. Below the map are indications of the approximate locations of regions of the glycoproteins, as follows, C1, C2, C3, C4=constant regions of gp120; V1/V2, V3, V4, V5=variable regions of gp120; gp41 regions=FP (fusion peptide), LZ (leucine zipper), AH (membrane proximal alpha helix), TM (transmembrane segment), and CT (cytoplasmic tail). (B) Schematic description of a series of chimeric genes (Chim) constructed by exchanging segments of the MN-TCLA and MN-P clones, as described in Materials and Methods. (C) Relative infectivity and neutralization titers obtained for the clones. Relative infectivity was determined as X_{M}/X_{MN-TCLA} where X is the virus dilution that yields a given activity. X_{M} is the value obtained for the particular mutant studied and X_{MN-TCLA} is the value obtained for MN-TCLA. Best-fit lines were determined by regression analysis of the log-transformed luciferase activity determinations (light units) as a function of inoculum dilutions. y values used for comparisons of MN-TCLA and other clones were selected so as to intersect approximately linear segments of the curves being compared. Neutralization phenotypes of chimeras were determined by using HIV Neutralizing Serum 2 (HNS2). Titers are the reciprocal serum dilutions resulting in ≧90% inhibition of infectivity. Each chimeric clone was tested 3-5 times in comparison to MN-P and MN-TCLA, and geometric mean results are presented.

FIG. 3. Schematic representations of recombinant env genes used for analysis of genetic basis for resistance to sCD4 neutralization. (A) Gene organization. (B) Structure of additional chimeric genes. Segments of each clone derived from MN-TCLA are shown as shaded bars, and segments derived from MN-P are shown as white bars. The nature of mutations introduced by site-directed mutagenesis is indicated, with locations noted by vertical lines.

FIG. 4. Neutralization by sCD4 of viruses pseudotyped with the envelopes MN-P (open squares), or Chimeras A (open circles), GC (closed squares), HC (closed diamonds), or C (closed circle). Neutralization sensitivities of MN-TCLA (not shown) and Chimera C were similar. Assays were performed in triplicate, and results shown are means obtained at each sCD4 concentration. Control luminescence was determined based on infections performed in the absence of sCD4. Essentially identical results were obtained in two similar experiments.

FIG. 5. Mutations in and around the CD4 binding site contribute to sCD4 resistance in the context of MN-P V1/V2 sequences. The relative neutralizing [sCD4] was determined for each clone as follows. The fifty percent inhibitory concentration (IC50) of sCD4 was determined by linear regression analysis. The relative neutralizing [CD4] was then calculated as Test clone IC50/Chimera C IC50. The IC50 for MN-P and for Chimera C were determined in each experiment. The number of assays for each clone is indicated in parentheses after the clone designation. The structures of Chimeras C, P, GC, and HC are illustrated in FIGS. 2 and 3. Individual clones were constructed that contained one or combinations of the seven CD4 binding domain mutations. Chimeras C/7CD4bs/V3, P/7CD4bs, and P/7CD4bs/V3 each contain the seven CD4 binding domain mutations from MN-P in the respective Chimeras. Chimeras C/V3, C/7CD4bs/V3/P/V3, and P/7CD4bs/V3 each contain the four MN-P V3 region mutations, shown in FIG. 3.

FIG. 6. Correlation between neutralization resistance and infectivity of viruses pseudotyped with mutant and chimeric MN strain envelope clones. The null hypothesis of no correlation was rejected with p<0.0001.

FIG. 7. Resistance of Chimera GC to neutralization by sCD4 depends upon sequences in V3, the mutation at residue 426 in the co-receptor binding site, and mutations in the outer domain distant from the CD4 and co-receptor binding sites. Mutations at residues 298, 345, 418, and 300, in the outer domain distant from the CD4 and co-receptor binding sites, were introduced sequentially into Chimera C/7CD4bs/V3. The V/I mutation at residue 426 was introduced into both Chimera C and Chimera C/7CD4bs/V3. Four mutations corresponding to the MN-TCLA V3 region sequence were introduced into Chimera GC to form Chimera GC-V3. Results shown are means of three assays, each done in triplicate. Statistical comparisons were done using ANOVA (C versus C/V3 versus C/7CD4bs/V3 versus C/7CD4bs/V3/398 versus C/7CD4bs/V3/345/398) or Student's t test (C/7CD4bs/V3/345/398/418 versus C/7CD4bs/V3/300/345/398/418; C/7CD4bs/V3/426 versus GC; and BC-V3 versus GC). Results shown are the means and standard deviations of three experiments, each done in triplicate.

FIG. 8. Functional interactions between V1/V2 and residues near the CD4 binding domain and V3 affect global neutralization resistance. The clones MN-P (closed bars), Chimera P/7CD4bs (hatched bars), and Chimera P/7CD4bs/V3 (open bars) were tested for neutralization. Results shown are averages of two assays, each done in triplicate. (A) Neutralization by the anti-CD4 binding domain ligands sCD4, CD4-IgG2, F105, F91, and b12. (B)Neutralization by antibodies directed against the co-receptor-binding site, 17b and 4.8d, and the V3 region, 19b. In this panel, the maximum concentration of each antibody used was 2.5 μg/1 ml. There was no neutralization of MN-P at any concentration, so the inhibitory concentration was considered to be ≧5 μg/ml.

FIG. 9. Localization of MN-P/MN-TCLA mutations in the atomic structure of HIV-1 gp120 core complexed with CD4 and a neutralizing antibody 17b. PDF file is from Protein Data Bank, 1GC1 (Kwong et al. (1998) Nature 393, 648-659). Drawn with PCMolecule2 (version 2.0.0) (Molecular Ventures). Front (A) and back (B) sides of molecular complex are shown. The gp120 is shown in blue, CD4 in green, and the 17b antibody in yellow (light chain) and in pale blue (heavy chain). Amino acid sequence of gp120 was extracted form PDF file and aligned with the sequence of MN-TCLA by ClustalW (version 1.7). Mutation sites are colored by red and marked as, MN-P a.a. and position number of mutation. Position numbers are represented with respect to a.a. positions in MN-TCLA sequence. Locations of V1/V2, V3, V4, and V5 loops are also indicated.

FIG. 10. Effects of inner domain and selected outer domain mutations on infectivity. Restriction enzyme cleavage map of MN-TCLA, shown at top, is described in FIG. 2. Schematic diagram of chimeric env genes constructed using fragments of the MN-TCLA and MN-P genes are shown on the left. Stars above the diagrams of chimeras (Chim) indicate the approximate locations of mutations introduced in vitro (see text for description). Relative infectivities of viruses pseudotyped with envelopes encoded by the chimeric genes are shown to the right.

FIG. 11. Particle association of gp120. (A) Results shown in Table 1. Particle associated fraction of gp120 (PAF gp120) was estimated as P/(P+S) for each clone, where P is the amount of gp120 in pellets and S and S is the amount in supernatants. The results are expressed as PAF (gp120)_{M}/PFA (gp120)_{TCLA}, where M refers to the mutant clone being tested. (B) The PAF (gp120) results are normalized for p24 distribution. PAF (p24) was calculated for each clone. PAF (gp120/p24) was calculated as the ratio of PAF (gp120)/PAF(p24). These results are shown as the ratio of PAF (gp120/p24)_{M}/PAF (gp120/p24)_{TCLA}.

FIG. 12. Schematic representation of the functional interactions of the V1/V2 and V3 regions and gp41 over the surface of gp120. The cysteine residues at the stalks of the V1/V2 and V3 loops are shown in magenta, and red arrows highlight their locations.

### DETAILED DESCRIPTION

Previously, the selection and characterization of a neutralization-resistant mutant of MN strain of HIV-1 was described (Park et al. (1998) J. Virol. 72, 7099-7107; Park et al. (1999) J. Virol. 73, 5707-5713; Park et al. (2000) J. Virol. 74, 4183-4191). The phenotype was attributable to two mutations in gp120, and four in the leucine zipper (LZ) structure of gp41. The neutralization-resistant phenotype was found to be associated with a high infectivity phenotype, which was attributable to five of the six mutations. The high infectivity is, in turn, the result of high efficiency of steps that follow cell surface binding of virus during infection, and that leads to virus-cell membrane fusion (Park et al. (2000) J. Virol. 74, 4183-4191). The present invention relates to HIV-1 envelope proteins that were developed from the extremely neutralization sensitive, T cell line adapted (TCLA), MN strain (MN-TCLA) and the neutralization resistant, primary MN strain (MN-P) of HIV-1. These phenotypes were dependent upon multiple mutations distributed throughout gp120 and gp41, and functional interactions of regions of gp120 with LZ sequences in gp41. Some of the mutations localize in or near gp120 binding sites for CD4 or co-receptor. The neutralization resistance of the primary HIV-1 strain is the result of multiple mutations that transduce effects throughout the envelope protein complex, conferring a high infection efficiency phenotype.

Thus, the present invention provides a method for the rational design and preparation of vaccines and immunogenic compositions based on HIV envelope proteins. This invention includes the discovery that certain amino acid residues in gp120 mediate functional interactions with gp41, CD4 and co-factor receptors (e.g., CCR5 an CXCR4). Although the amino acid sequences of the CD4, co-receptor and gp41 binding domains in gp120 are variable, it has now been determined that substitution of certain amino acid residues can effect the ability to the envelope protein to induce a broadly cross-reactive immune response. This facilitates the design of an HIV subunit vaccine or immunogenic composition that can induce antibodies that neutralize HIV strains across different phenotypes and clades.

### Methods for Identifying HIV-1 Envelope Proteins

The invention encompasses a method of identifying a human immuno-deficiency virus type-1 (HIV-1) envelope protein which produces a cross-reactive immune response following administration in a mammal (e.g., human). The method of this invention is based in part on the discovery that there are amino acid residues in or near the CD4, gp41 and co-factor receptor binding domains of gp120 critical for sensitivity to antibody neutralization. These residues are located across multiple domains in gp120 including C2, C3, C4 or V5. These residues can also be located in the C1, C5, V1/V2, V3 or V4 domains of gp120 or any domain of gp41 (e.g. leucine zipper domain).

Identifying and substituting the appropriate amino acids in an HIV-1 envelope protein in any of these domains provides a vaccine or immunogenic composition that is designed to produce a broadly cross-reactive immune response against different strains of HIV-1 even across different clades. Although the amino acid sequences of these domains containing these amino acids is variable, the position of these residues in the gp41, CD4 and co-receptor binding domains is conserved, facilitating the design of a vaccine or immunogenic composition which can neutralize a plurality of the most common HIV strains across different clades (e.g., A, A1, A2, B, C, D, F, F1, F2, G, H, J, K, N, O, V).

The first step in identifying an envelope protein capable of generating a broadly cross-reactive immune response is to determine the location and type of the amino acids in the gp120 domains which interact with gp41, CD4 and co-receptors or the outer domain which comprises amino acids exposed on the surface of the gp120 protein. These locations can be determined by sequencing the region of gp120 containing these amino acids. Alternatively, when antibodies specific for any of these binding domains are available, preferably monoclonal antibodies, the effect of substitution of these amino acids at different locations can be determined by serological methods as described hereinafter.

In one embodiment, these amino acids are in the one or more of the C2, C3, C4 or V5 domains while in other embodiments these amino acids are located in one or more of the C1, C5, V1/V2, V3 or V4 domains. The location of these amino acid residues in the gp120 protein include, but are not limited to, amino acids corresponding to positions 64, 84, 91, 219, 243, 245, 249, 287, 290, 300, 211, 312, 314, 316, 345, 371, 372, 398, 426, 418, 435, 466 and 472 of SEQ ID NO: 2 or 4. Examples of these substitutions include but are not limited to V64A, Q84E, N243K, T245S, P249S, D287N, D290N, N311Y, Y312N, K314R, K316T, N371K, P372Q, V426I, K435N, E466N and D472N. The role of each of the amino acids in these positions, along with appropriate substitutions is described in detail in the Examples.

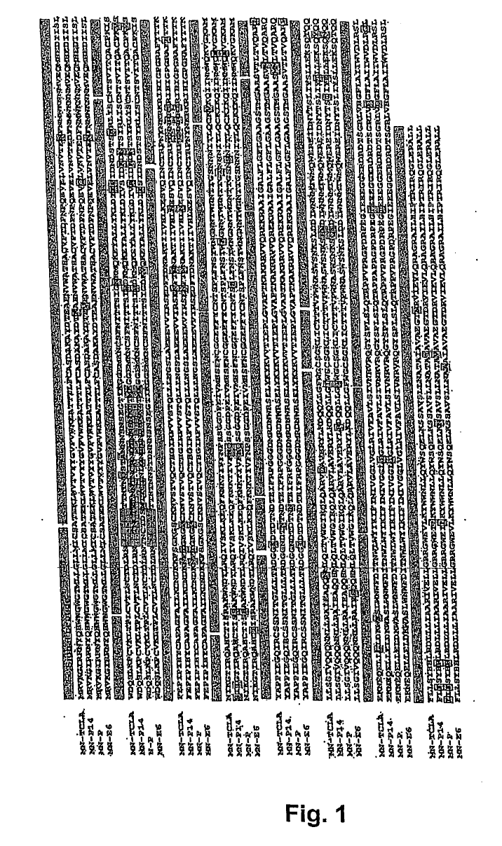

When discussing the amino acid sequences of various isolates and strains of HIV, the most common numbering system refers to the location of amino acids within the gp120 protein using the initiator methionine residue as position 1. The amino acid numbering reflects the mature HIV-1 gp120 amino acid sequence as shown, for example, in the alignment in FIG. 1. For gp120 sequences derived from other HIV isolates and which include their native N-terminal signal sequence, numbering may differ. Although the nucleotide and amino acid residue numbers may not be consistent in other strains where upstream deletions or insertions change the length of the viral genome and gp120, the region corresponding regions or individual amino acid residues are readily identified by reference to the teachings herein. The variable (V) domains and conserved (C) domains of gp120 are specified according to the nomenclature of Modrow et al. (1987) J. Virol. 61, 570-578 which is hereby incorporated by reference in its entirety.

For identifying the effect of any amino acid substitution in an HIV-1 envelope protein on it neutralization phenotype, an animal is immunized with modified gp120 to induce anti-gp120 antibodies. The antibodies can be polyclonal or monoclonal. Methods for the preparation of immunogenic compositions of a protein may vary depending on the host animal and the protein and are well known. For example, gp120 or an antigenic portion thereof can be conjugated to an immunogenic substance such as KLH or BSA or provided in an adjuvant or the like. The induced antibodies can be tested to determine whether they are specific for gp120. If a polyclonal antibody composition does not provide the desired specificity, the antibodies can be fractionated by ion exchange chromatography and immunoaffinity methods using intact gp120 or various fragments of gp120 to enhance specificity by a variety of conventional methods. For example, the antibody composition can be fractionated to reduce binding to other substances by contacting the composition with gp120 affixed to a solid substrate. Those antibodies which bind to the substrate are retained. Fractionation techniques using antigens affixed to a variety of solid substrates such as affinity chromatography materials including Sephadex, Sepharose and the like are well known.

Monoclonal anti-gp120 antibodies can be produced by a number of conventional methods. A mouse can be injected with an immunogenic composition containing gp120 and spleen cells obtained. Those spleen cells can be fused with a fusion partner to prepare hybridomas. Antibodies secreted by the hybridomas can be screened to select a hybridoma wherein the antibodies neutralize HIV infection, as described herein. Hybridomas that produce antibodies of the desired specificity are cultured by standard techniques.

Infected human lymphocytes can be used to prepare human hybridomas by a number of techniques such as fusion with a murine fusion partner or transformation with EBV. In addition, combinatorial libraries of human or mouse spleen can be expressed in E. coli to produce the antibodies. Kits for preparing combinatorial libraries are commercially available. Hybridoma preparation techniques and culture methods are well known and constitute no part of the present invention.

Following preparation of anti-gp120 monoclonal antibodies, the antibodies are screened to determine those antibodies which are neutralizing antibodies. Assays to determine whether a monoclonal antibody neutralizes HIV infection are well known and are described in the literature. Briefly, dilutions of antibody and HIV stock are combined and incubated for a time sufficient for antibody binding to the virus. Thereafter, cells that are susceptible to HIV infection are combined with the virus/antibody mixture and cultured. MT-2 cells or H9 cells are susceptible to infection by most HIV strains that are adapted for growth in the laboratory. Activated peripheral blood mononuclear cells (PBMC) or macrophages can be infected with primary isolates (isolates from a patient specimens which have not been cultured in T-cell lines or transformed cell lines). Daar et al. (1990) Proc. Natl. Acad. Sci. USA 87, 6574-6578 describe methods for infecting cells with primary isolates.

After culturing the cells for about five days, the number of viable cells is determined, by measuring metabolic conversion of the formazan MTT dye. The percentage of inhibition of infectivity is calculated to determine those antibodies that neutralize HIV. An exemplary preferred procedure for determining HIV neutralization is described in the Examples.

Those monoclonal antibodies which neutralize HIV are used to map the epitopes containing the amino acid substitutions to which the antibodies bind. To determine the location of a gp120 neutralizing epitope, neutralizing antibodies may be combined with fragments of gp120 to determine the fragments to which the antibodies bind. The gp120 fragments used to localize the neutralizing epitopes are preferably made by recombinant DNA methods as described herein and exemplified in the Examples. By using a plurality of fragments, each encompassing different, overlapping portions of gp120, an amino acid sequence encompassing a neutralizing epitope to which a neutralizing antibody binds can be determined.

This use of overlapping fragments can narrow the location of the epitope to a region of about 20 to 40 amino acid residues. To confirm the location of the epitope and narrow the location to a region of about 5 to 10 residues, site-directed mutagenicity studies are performed. Such studies can also determine the critical residues for binding of neutralizing antibodies.

In addition to antibodies, soluble CD4 receptor can be used to assess sensitivity to neutralization. As used herein, soluble CD4 receptor includes the entire human CD4 receptor protein and fragments thereof. In some embodiments, the soluble CD4 receptor fragments lacks the transmembrane domain as is well known in the art and provided in the Examples herein.

To perform site-directed mutagenicity studies, recombinant PCR techniques can be utilized to introduce single amino acid substitutions at selected sites into gp120 fragments containing the neutralizing epitope. Briefly, overlapping portions of the region containing the epitope are amplified using primers that incorporate the desired nucleotide changes. The resultant PCR products are annealed and amplified to generate the final product. The final product is then expressed to produce a mutagenized gp120 fragment. Expression of DNA encoding gp120 or a portion thereof is described herein and exemplified in the Examples.

Modified and/or chimeric gp120 proteins are then used in an immunoassay using gp120 as a control to determine when the mutations impair or eliminate binding of neutralizing antibodies. Those critical amino acid residues form part of a neutralizing epitope that can only be altered in limited ways without eliminating the epitope. Each alteration that preserves the epitope can be determined. Such mutagenicity studies demonstrate the variations in the amino acid sequence of the neutralizing epitope that provide equivalent, diminished or enhanced binding by neutralizing antibodies or eliminate antibody binding. Alterations in the amino acid sequence of neutralizing epitope that are suitable for use in a vaccine or an immunogenic composition can be determined by such an analysis.

Alternatively, the nucleotide sequence of DNA encoding gp120 or a relevant portion of gp120 can be determined and the amino acid sequence of gp120 can be deduced. Methods for amplifying gp120-encoding DNA from HIV isolates to provide sufficient DNA for sequencing are well known. In particular, Ou et al. (1992) Science 256, 1165-1171; Zhang et al. (1991) AIDS 5, 675-681 and Wolinsky (1992) Science 255, 1134-1137 describe methods for amplifying gp120 DNA. Sequencing of the amplified DNA is well known and is described in Sambrook et al. (2001) Molecular Cloning: A Laboratory Manual, Cold Spring Harbor Laboratory Press.

Alignment of envelope amino acid and nucleotide sequences to identify the location of the amino acids to be substituted in any particular envelope protein can be accomplished by BLAST (Basic Local Alignment Search Tool) analysis using the algorithm employed by the programs blastp, blastn, blastx, tblastn and tblastx (Karlin et al. (1990) Proc. Natl. Acad. Sci. USA 87, 2264-2268 and Altschul, (1993) J. Mol. Evol. 36, 290-300, fully incorporated by reference). The approach used by the BLAST program is to first consider similar segments between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al. (1994) Nature Genet. 6, 119-129) which is fully incorporated by reference. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al. (1992) Proc. Natl. Acad. Sci. USA 89, 10915-10919, fully incorporated by reference). Four blastn parameters were adjusted as follows: Q=10 (gap creation penalty); R=10 (gap extension penalty); wink=1 (generates word hits at every wink^{th} position along the query); and gapw=16 (sets the window width within which gapped alignments are generated). The equivalent Blastp parameter settings were Q=9; R=2; wink=1; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

### HIV-1 Envelope Proteins and Peptides

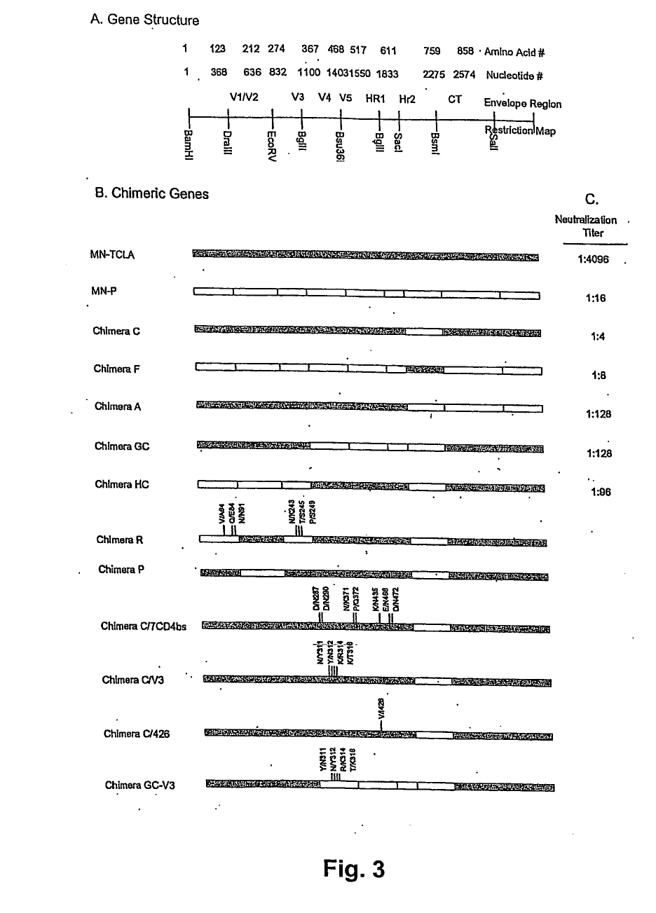

HIV-1 envelope proteins and peptides of the invention include envelope proteins having one or more of any of the amino acid substitutions set for in FIG. 3. These substitutions can be in one or multiple domains of the envelope protein including V1/V2, V3, V4, V5, C1, C2, C3, C4 and C5. These include one or more amino acid substitutions at a position corresponding to an amino acid in gp120 selected from the group consisting of amino acids 64, 84, 91, 219, 243, 245, 249, 287, 290, 300, 211, 312, 314, 316, 345, 371, 372, 398, 426, 418, 435, 466 and 472. Substitutions at these locations have been determined to effect interactions of gp120 with gp41, CD4 and co-receptors and subsequently sensitivity to neutralization by antibodies.

The HIV-1 envelope proteins of the invention may also be chimeric envelope proteins. As used herein, a “chimeric envelope protein” refers to an envelope protein containing at least one first domain or fragment of amino acid sequence substituted for the corresponding sequence or fragment in a second envelope protein. Generally, these chimeric envelope proteins are produced by recombinant methods well known in the art and therefore do not occur naturally. The size of the domain or amino acid fragment from the first domain can range from about 5 to about 800 amino acids. In one embodiment, the invention includes an isolated HIV-1 envelope protein comprising at least one first fragment of an antibody neutralization-resistant envelope protein and at least one second fragment from a antibody neutralization-sensitive envelope protein. In some embodiments, the first fragment is derived from an antibody neutralization-resistant envelope protein such as MN-P (SEQ ID NO: 2) while the second fragment of a antibody neutralization-sensitive envelope protein is derived from MN-TCLA (SEQ ID NO: 4). Examples of domains or amino acid fragments from envelope proteins that can be substituted into a second envelope protein include but are not limited to, amino acids about 1 to about 123, about 123 to about 212, about 212 to about 274, about 274 to about 367, about 367 to about 468, about 468 to about 517, about 517 to about 611, about 611 to about 759, and about 759 to about 858.

In some embodiments, the chimeric HIV-1 envelope further comprises one or more amino acid substitutions. These substitutions can be in one or multiple domains of the envelope protein including V1/V2, V3, V4, V5, C1, C2, C3, C4 and C5. These include one or more amino acid substitutions at a position corresponding to an amino acid in gp120 selected from amino acids 64, 84, 91, 219, 243, 245, 249, 287, 290, 300, 211, 312, 314, 316, 345, 371, 372, 398, 426, 418, 435, 466 and 472. Examples of these substitutions include but are not limited to V64A, Q84E, N243K, T245S, P249S, D287N, D290N, N311Y, Y312N, K314R, K316T, N371K, P372Q, V4261, K435N, E466N and D472N.

The envelope proteins of the present invention may be prepared by any known techniques including recombinant methods as described herein. In addition, the peptides may be prepared using solid-phase synthetic techniques well known in the art. Examples of peptide synthesis techniques may be found, for example, in Bodanszky et al. (1976) Peptide Synthesis, Wiley.

As used herein, a envelope protein is said to be “isolated” when the protein is substantially separated from other contaminants including envelope proteins from different HIV.

### Nucleic Acids and Recombinant Protein Expression

HIV-1 envelope proteins of the invention may be prepared by any available means, including recombinant expression of the desired protein or peptide in eukaryotic or prokaryotic host cells (see U.S. Pat. No. 5,696,238). Methods for producing proteins of the invention for purification may employ conventional molecular biology, microbiology, and recombinant DNA techniques within the ordinary skill level of the art. Such techniques are explained fully in the literature (see, for example, Sambrook et al. (2001) Molecular Cloning: A Laboratory Manual, Cold Spring Harbor Laboratory Press; Glover (1985) DNA Cloning: A Practical Approach, IRL Press).

The present invention further provides nucleic acid molecules that encode the HIV-1 envelope proteins of the invention. Such nucleic acid molecules can be in an isolated form, or can be operably linked to expression control elements or vector sequences. The present invention further provides host cells that contain the vectors via transformation, transfection, electroporation or any other art recognized means of introducing a nucleic acid into a cell.

As used herein, a “replicon” is any genetic element (e.g., plasmid, chromosome, virus) that functions as an autonomous unit of DNA replication in vivo (i.e., capable of replication under its own control).

As used herein, a “vector” is a replicon, such as plasmid, phage or cosmid, to which another nucleic acid (e.g., DNA) segment may be attached so as to bring about the replication of the attached segment. Vectors of the invention include viral vectors.

As used herein, a “nucleic acid” refers to the polymeric form of ribonucleotide or deoxyribonucleotides (adenine, guanine, thymine, and/or cytosine) in either its single stranded form, or in double-stranded helix. This term refers only to the primary and secondary structure of the molecule and is not limited to any particular tertiary form. Thus, this term includes single-stranded RNA or DNA, double-stranded DNA found in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the non-transcribed strand of DNA (e.g., the strand having a sequence homologous to the mRNA).

A nucleic acid “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxy) terminus. A polyadenylation signal and transcription termination sequence will usually be located 3′ to the coding sequence.

Transcriptional and translational control sequences are DNA regulatory sequences, such as promoters, enhancers, polyadenylation signals, terminators, and the like, that provide for the expression of a coding sequence in a host cell.

As used herein, a “promoter sequence” is a DNA regulatory region capable of binding RNA polymerase in a cell and initiating transcription of a downstream (3′ direction) coding sequence. For purposes of defining the present invention, the promoter sequence is bounded (inclusively) at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Eukaryotic promoters will often, but not always, contain “TATA” boxes and “CAT” boxes.

A coding sequence is “under the control” of transcriptional and translational control sequences in a cell when RNA polymerase transcribes the coding sequence into mRNA, which is then translated into the protein encoded by the coding sequence.

A “signal sequence” can be included before the coding sequence or the native 29 amino acid signal peptide sequence from an envelope protein may be used. This sequence encodes a signal peptide, N-terminal to the polypeptide, that communicates to the host cell to direct the polypeptide to the cell surface or secrete the polypeptide into the media. This signal peptide is clipped off by the host cell before the protein leaves the cell. Signal sequences can be found associated with a variety of proteins native to prokaryotes and eukaryotes. For instance, alpha-factor, a native yeast protein, is secreted from yeast, and its signal sequence can be attached to heterologous proteins to be secreted into the media (see U.S. Pat. No. 4,546,082). Further, the alpha-factor and its analogs have been found to secrete heterologous proteins from a variety of yeast, such as Saccharomyces and Kluyveromyces (EP 88312306.9; EP 0324274 publication, and EP 0301669). An example for use in mammalian cells is the tPA signal used for expressing Factor vIIIc light chain.

A cell has been “transformed” by a exogenous or heterologous nucleic acid when such nucleic acid as been introduced inside the cell. The transforming nucleic acid may or may not be integrated (covalently linked) into chromosomal DNA malting up the genome of the cell. In prokaryotes, for example, the transforming nucleic acid may be maintained on an episomal element such as a plasmid or viral vector. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming nucleic acid.

As used herein, a “cell line” is a clone of a primary cell that is capable of stable growth in vitro for many generations. As used herein, nucleic acid sequences are “substantially homologous” when at least about 85% (preferably at least about 90% and most preferably at least about 95%) of the nucleotides match over the defined length of the DNA sequences. Sequences that are substantially homologous can be identified in a Southern hybridization experiment under, for example, stringent conditions as defined for that particular system. Defining appropriate hybridization conditions is within the skill of the art.

A “heterologous” region of the nucleic acid construct is an identifiable segment of a nucleic acid within a larger nucleic acid molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene).

Vectors are used to simplify manipulation of the nucleic acids which encode the HIV envelope proteins or peptides, either for preparation of large quantities of nucleic acids for further processing (cloning vectors) or for expression of the HIV envelope proteins of peptides (expression vectors). Vectors comprise plasmids, viruses (including phage), and integrated DNA fragments (i.e., fragments that are integrated into the host genome by recombination). Cloning vectors need not contain expression control sequences. However, control sequences in an expression vector include transcriptional and translational control sequences such as a transcriptional promoter, a sequence encoding suitable ribosome binding sites, and sequences which control termination of transcription and translation. The expression vector should preferably include a selection gene to facilitate the stable expression of HIV envelope gene and/or to identify transformed cells. However, the selection gene for maintaining expression can be supplied by a separate vector in co-transformation systems using eukaryotic host cells.

Suitable vectors generally will contain replicon (origins of replication, for use in non-integrative vectors) and control sequences which are derived from species compatible with the intended expression host. By the term “replicable” vector as used herein, it is intended to encompass vectors containing such replicons as well as vectors which are replicated by integration into the host genome. Transformed host cells are cells which have been transformed or transfected with vectors containing HIV envelope peptide or protein encoding nucleic acid. The expressed HIV envelope proteins or peptides may be secreted into the culture supernatant, under the control of suitable processing signals in the expressed peptide (e.g. homologous or heterologous signal sequences).

Expression vectors for host cells ordinarily include an origin of replication, a promoter located upstream from the HIV envelope protein or peptide coding sequence, together with a ribosome binding site, a polyadenylation site, and a transcriptional termination sequence. Those of ordinary skill will appreciate that certain of these sequences are not required for expression in certain hosts. An expression vector for use with microbes need only contain an origin of replication recognized by the host, a promoter which will function in the host, and a selection gene.

Commonly used promoters are derived from polyoma, bovine papilloma virus, CMV (cytomegalovirus, either murine or human), Rouse sarcoma virus, adenovirus, and simian virus 40 (SV40). Other control sequences (e.g., terminator, polyA, enhancer, or amplification sequences) can also be used.

An expression vector is constructed so that the HIV-1 envelope protein or peptide coding sequence is located in the vector with the appropriate regulatory sequences, the positioning and orientation of the coding sequence with respect to the control sequences being such that the coding sequence is transcribed and translated under the “control” of the control sequences (i.e., RNA polymerase which binds to the DNA molecule at the control sequences transcribes the coding sequence). The control sequences may be ligated to the coding sequence prior to insertion into a vector, such as the cloning vectors described above. Alternatively, the coding sequence can be cloned directly into an expression vector which already contains the control sequences and an appropriate restriction site. If the selected host cell is a mammalian cell, the control sequences can be heterologous or homologous to the HIV-1 envelope protein coding sequence, and the coding sequence can either be genomic DNA containing introns or cDNA.

Higher eukaryotic cell cultures may be used to express the proteins of the present invention, whether from vertebrate or invertebrate cells, including insects; and the procedures of propagation thereof are known.

It will be appreciated that when expressed in mammalian tissue, the recombinant HIV gene products may have higher molecular weights than expected due to glycosylation. It is therefore intended that partially or completely glycosylated forms of HIV envelope pre-proteins or peptides having molecular weights somewhat different from 160, 120 or 41 kiloDaltons are within the scope of this invention.

Other expression vectors are those for use in eukaryotic systems. An exemplary eukaryotic expression system is that employing vaccinia virus, which is well-known in the art (see, for example, WO 86/07593). Yeast expression vectors are known in the art (see, for example, U.S. Pat. Nos. 4,446,235 and 4,430,428). Another expression system is vector pHSI, which transforms Chinese hamster ovary cells (see WO 87/02062). Mammalian tissue may be cotransformed with DNA encoding a selectable marker such as dihydrofolate reductase (DHFR) or thymidine kinase and DNA encoding the HIV envelope protein or peptide. If wild type DHFR gene is employed, it is preferable to select a host cell which is deficient in DHFR, thus permitting the use of the DHFR coding sequence as marker for successful transfection in hgt medium, which lacks hypoxanthine, glycine, and thymidine.

Depending on the expression system and host selected, HIV envelope proteins or peptides are produced by growing host cells transformed by an exogenous or heterologous DNA construct, such as an expression vector described above under conditions whereby the HIV envelope protein is expressed. The HIV protein or peptide is then isolated from the host cells and purified. If the expression system secretes the protein or peptide into the growth media, the protein can be purified directly from cell-free media. The selection of the appropriate growth conditions and initial crude recovery methods are within the skill of the art.

Once a coding sequence for an HIV envelope protein or peptide of the invention has been prepared or isolated, it can be cloned into any suitable vector and thereby maintained in a composition of cells which is substantially free of cells that do not contain any HIV envelope protein coding sequence. As described above, numerous cloning vectors are known to those of skill in the art.

### Vaccine Compositions

When used in vaccine or immunogenic compositions, the HIV envelope proteins of the present invention may be used as “subunit” or other vaccines or immunogens. Such vaccines or immunogens offer significant advantages over traditional vaccines in terms of safety and cost of production; however, subunit vaccines are often less immunogenic than whole-virus vaccines, and it is expected that adjuvants with significant immunostimulatory capabilities may be added in order to reach their full potential.

The term “subunit vaccine” is used herein, as in the art, to refer to a viral vaccine that does not contain virus, but rather contains one or more viral proteins or fragments of viral proteins. As used herein, the term “multivalent” means that the vaccine contains modified gp120 from at least two different HIV-1 isolates.

Currently, adjuvants approved for human use in the United States include aluminum salts (alum). These adjuvants have been useful for some vaccines including hepatitis B, diphtheria, polio, rabies, and influenza. Other useful adjuvants include Complete Freund's Adjuvant (CFA), Incomplete Freund's Adjuvant (IFA), Muramyl dipeptide (MDP), synthetic analogues of MDP, N-acetylnuramyl-L-alanyl-D-isoglutamyl-L-alanine-2-[1,2-dipalmitoyl-sn-glycero-3-(hydroxy-phosphoryloxy)]ethylamide (MTP-PE) and compositions containing an oil capable of being metabolized and an emulsifying agent, wherein the oil and emulsifying agent are present in the form of an oil-in-water emulsion having oil droplets substantially all of which are less than one micron in diameter (see, for example, EP 0399843).

The formulation of a vaccine or immunogenic compositions of the invention will employ an effective amount of the protein or peptide antigen. That is, there will be included an amount of antigen which, in combination with the adjuvant, will cause the subject to produce a specific and sufficient immunological response so as to impart protection to the subject from subsequent exposure to any type of HIV. When used as an immunogenic composition, the formulation will contain an amount of antigen which, in combination with the adjuvant, will cause the subject to produce specific antibodies which may be used for diagnostic or therapeutic purposes.

The vaccine compositions of the invention may be useful for the prevention or therapy of HIV-1 infection. While all animals that can be afflicted with HIV-1 can be treated in this manner, the invention, of course, is particularly directed to the preventive and therapeutic use of the vaccines of the invention in man. Often, more than one administration may be required to bring about the desired prophylactic or therapeutic effect; the exact protocol (dosage and frequency) can be established by standard clinical procedures.

The vaccine compositions are administered in any conventional manner which will introduce the vaccine into the animal, usually by injection. For oral administration the vaccine composition can be administered in a form similar to those used for the oral administration of other proteinaceous materials, such as insulin. As discussed above, the precise amounts and formulations for use in either prevention or therapy can vary depending on the circumstances of the inherent purity and activity of the antigen, any additional ingredients or carriers, the method of administration and the like.

By way of non-limiting illustration, the vaccine dosages administered will typically be, with respect to the gp120 antigen, a minimum of about 0.1 mg/dose, more typically a minimum of about 1 mg/dose, and often a minimum of about 10 mg/dose. The maximum dosages are typically not as critical. Usually, however, the dosage will be no more than about 1 mg/dose, typically no more than 500 mg/dose, often no more than 250 mg/dose. These dosages can be suspended in any appropriate pharmaceutical vehicle or carrier in sufficient volume to carry the dosage. Generally, the final volume, including carriers, adjuvants, and the like, typically will be at least 0.1 ml, more typically at least about 0.2 ml. The upper limit is governed by the practicality of the amount to be administered, generally no more than about 0.5 ml to about 1.0 ml.

Peptides of the invention corresponding to domains of the envelope protein such as any of the CD4, gp41 or co-factor binding domains of gp120 may be constructed or formulated into compounds or compositions comprising multimers of the same domain or multimers of different domains. For instance, peptides corresponding to the V5 domain may be circularized by oxidation of the cysteine residues to form multimers containing 1, 2, 3, 4 or more individual peptide epitopes. The circularized form may be obtained by oxidizing the cysteine residues to form disulfide bonds by standard oxidation procedures such as air oxidization.

Synthesized peptides of the invention may be circularized in order to mimic the geometry of those portions as they occur in the envelope protein. Circularization may be facilitated by disulfide bridges between existing cysteine residues. Cysteine residues may also be included in positions on the peptide which flank the portions of the peptide which are derived from the envelope protein. Alternatively, cysteine residues within the portion of a peptide derived from the envelope protein may be deleted and/or conservatively substituted to eliminate the formation of disulfide bridges involving such residues. Other means of circularizing peptides are also well known. The peptides may be circularized by means of covalent bonds, such as amide bonds, between amino acid residues of the peptide such as those at or near the amino and carboxy termini (see, for example, U.S. Pat. No. 4,683,136).

In another format, vaccine or immunogenic compositions may be prepared as vaccine vectors which express the HIV envelope protein or peptide of the invention in the host animal. Any available vaccine vector may be used, including Vaccina virus, Venezuelan Equine Encephalitis virus replicons (see, for example, U.S. Pat. No. 5,643,576). Alternatively, naked nucleic acid encoding a protein or peptide of the invention may be administered directly to effect expression of the antigen (see, for example, U.S. Pat. No. 5,739,118).

Preparation of gp120 for use in a vaccine is well known and is described hereinafter. With the exception of the use of the modified envelope protein, the vaccine prepared in the method need not differ from gp120 subunit vaccines of the prior art.

As with prior art gp120 subunit vaccines, gp120 at the desired degree of purity and at a sufficient concentration to induce antibody formation is mixed with a physiologically acceptable carrier. A physiologically acceptable carrier is nontoxic to a recipient at the dosage and concentration employed in the vaccine. Generally, the vaccine is formulated for injection, usually intramuscular or subcutaneous injection. Suitable carriers for injection include sterile water, but preferably are physiologic salt solutions, such as normal saline or buffered salt solutions such as phosphate buffered saline or ringer's lactate. The vaccine generally contains an adjuvant. Useful adjuvants include QS21 which stimulates cytotoxic T-cells and alum (aluminum hydroxide adjuvant). Formulations with different adjuvants which enhance cellular or local immunity can also be used.

Addition excipients that can be present in the vaccine include low molecular weight polypeptides (less than about 10 residues), proteins, amino acids, carbohydrates including glucose or dextrans, chelating agents such as EDTA, and other excipients.

The vaccine can also contain other HIV proteins. In particular, gp41 or the extracellular portion of gp41 can be present in the vaccine. Since gp41 has a conserved amino acid sequence, the gp41 present in the vaccine can be from any HIV isolate. gp160 from an isolate used in the vaccine can replace gp120 in the vaccine or be used together with gp120 from the isolate. Alternatively, gp160 from an isolate having a different neutralizing epitope than those in the vaccine isolates can additionally be present in the vaccine.

Vaccine formulations generally include a total of about 300 to 600 μg of gp120, conveniently in about 1.0 ml of carrier. The amount of gp120 for any isolate present in the vaccine will vary depending on the immunogenicity of the gp120. Methods of determining the relative amount of an immunogenic protein in multivalent vaccines are well known and have been used, for example, to determine relative proportions of various isolates in multivalent polio vaccines.

The vaccines of this invention may be administered in the same manner as prior art HIV gp120 subunit vaccines. In particular, the vaccines are generally administered at zero, one, six, eight or twelve months, depending on the protocol. Following the immunization procedure, annual or bi-annual boosts can be administered. However, during the immunization process and thereafter, neutralizing antibody levels can be assayed and the protocol adjusted accordingly.

The vaccine may be administered to uninfected individuals. In addition, the vaccine can be administered to seropositive individuals to augment immune response to the virus, as with prior art HIV vaccines. It is also contemplated that DNA encoding the strains of gp120 for the vaccine can be administered in a suitable vehicle for expression in the host. In this way, gp120 can be produced in the infected host, eliminating the need for repeated immunizations.

Without further description, it is believed that one of ordinary skill in the art can, using the preceding description and the following illustrative examples, make and utilize the compounds of the present invention and practice the claimed methods. The following working examples describe embodiments of the present invention, and are not to be construed as limiting in any way the remainder of the disclosure.

### EXAMPLES

### Example 1

### Pseudotyped Virus Construction, Infectivity Titration and Neutralization Assays

Viruses pseudotyped with envelope glycoproteins derived form various env plasmids were constructed using pSV7d-env and pNL-Luc-E-R- as described previously (Park et al. (1998) J. Virol. 72, 7099-7107; Conner et al. (1996) J. Virol. 70, 5306-5311). The env plasmid DNA and pNL-Luc-E-R- were introduced into 50% confluent 293T cells by calcium phosphate transfection (Promega). The culture medium was replaced with fresh medium containing 1 μM sodium butyrate at eighteen hours post transfection. At forty-eight hours after transfection, the pseudotyped virus-containing supernatants were harvested, filtered through 45-μm-pore size filters, and used immediately for infectivity and neutralization assays. Alternatively, filtered pseudotyped viruses, supplemented with additional fetal bovine serum to a final concentration of 20% were stored at −80° C. To measure the infectivity of pseudotyped viruses, a luminescence assay with HOS-CD4⁺-CXCR4⁺ cells was used. Cells (1.5×10⁴/ml) were inoculated with serially diluted pseudotyped virus in 96-well plates with U-bottom wells. The cultures were incubated for three days at 37° C., after which the cells were washed with cold phosphate-buffered saline and lysed with 15 μl of lysis buffer (Promega). Luciferase activity was read in a Luminoscan luminometer (Labsystem).

Luminescence resulting from infections was expressed as a ratio in comparison to MN-TCLA. Relative infectivity was determined as X_{M}/X_{MN-TCLA} for a given y, calculated using the best-fit lines determined by regression analysis of the log-transformed luciferase activity determinations (light units) as a function of the inoculum dilutions. The neutralization phenotype of each pseudotyped virus was tested in a manner similar to that of the infectivity assay, except that 25-μl aliquots of serially diluted sera were mixed with equal volumes of the appropriate pseudotyped viruses and incubated for one hour at 4° C., after which HOS-CD4⁺-CXCR4⁺ cells were added. The pseudotyped virus dilutions were selected to produce luminescence in the presence of nonimmune serum of about one-hundred times the background. The neutralization endpoints were considered to be the highest serum dilution calculated to cause a reduction of luminescence by 90% compared to the nonneutralized control. Human Neutralizing Serum 2 (HNS2) and the Negative Reference Serum were used as reference neutralizing and non-neutralizing sera, respectively (National Institutes of Health AIDS Research and Reference Reagent Program (ARRRP 1983 and 2411), Vujcic et al. (1995) AIDS Res. Hum. Retroviruses 11, 783-787).

### Example 2

### Construction of Chimeric Envelope Glycoprotein Genes

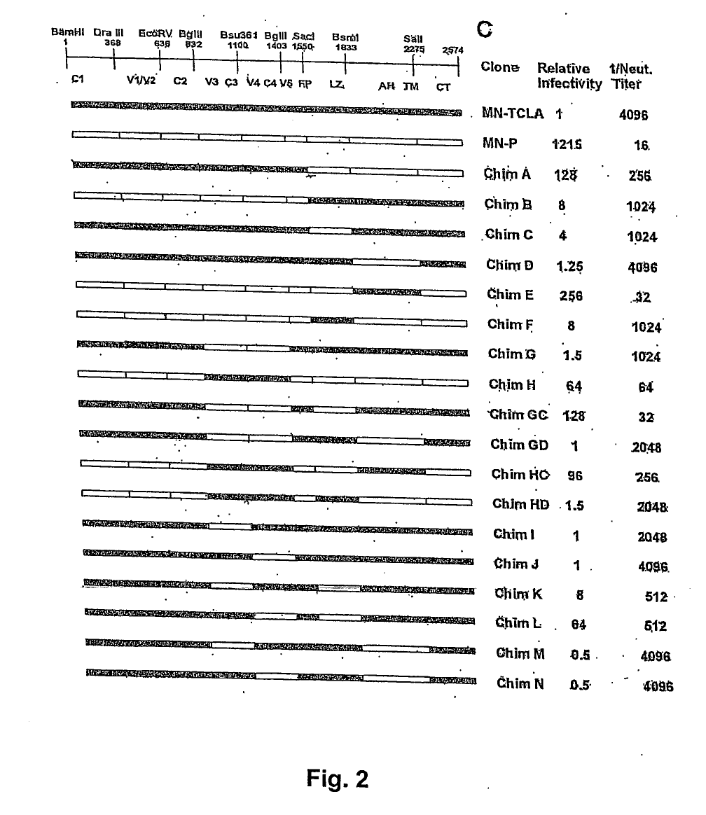

Several chimeric env clones were constructed by exchanging fragments of the neutralization sensitive, MN-TCLA, and the neutralization resistant, MN-P parental env clones, as previously described (Park et al. (1998) J. Virol. 72, 7099-7107). All chimeric env genes cloned into pSV7d vector were sequenced by the ABI PRISM big dye-terminator method (Applied Biosystems 3100 Genetic Analyzer). Analyses were performed using the EditSeq and MegAlign programs (DNAStar). The specific restriction enzymes and the locations of their recognition sequences are shown in FIG. 2. The nucleotide positions are numbered based on MNCG sequence (Gurgo et al. (1988) Virology 164, 531-536). Chimeras A and B were constructed by digesting the plasmids with Eco RI (upstream of the env start codon in pSV7d) and Sac I (nucleotide 1550). Chimera A contains the entire sequence of gp120 from MN-TCLA with the rest of the region (gp41) from MN-P, since the Sac I site is located 4 amino acids downstream from the cleavage site between gp120 and gp 41.

Chimeras C, D, E and F were constructed by initially subcloning the SacI-SalI fragments (725 nucleotides) at nucleotides 1550 and 2275 of both MN-TCLA and MN-P into pUC19. The products were called pUC19/MN-TCLAenv or pUC19/P37env, respectively. To construct chimera C, the SacI-BsmI fragment (267 nucleotides) of pUC19/P37env was ligated with the BsmI-SacI fragment of pUC19/MN-TCLAenv. The product was called pUC19/envC. The SacI-SalI fragment (725 nucleotides) of pUC19/envC was then ligated with the large SacI-SalI fragment of MN-TCLA (4493 nucleotides). To make chimera D, the SacI-BsmI fragment (267 nucleotides) of pUC19/MN-TCLAenv was ligated with the BsmI-SacI fragment of pUC19/P37env. The product was called pUC19/envD. The SacI-SalI fragment (725 nucleotides) of pUC19/envD was then ligated with the large SacI-SalI fragment of MN-TCLA (4493 nucleotides). To make chimera E, the SacI-SalI fragment (725 nucleotides) of pUC19/envD was ligated with the large SacI-SalI fragment of MN-P (4514nucleotides). To make chimera F, the SacI-SalI fragment (725 nucleotides) of pUC19/envD was ligated with the large SacI-SalI fragment of MN-P (4514 nucleotides). Chimeras G and H were constructed by exchanging large and small BglII-BglII fragments 571 and 4668 nucleotides, respectively, between MN-P and MN-TCLA. To construct chimera GC, the SacI-SalI fragment (725 bp) of Chimera C was ligated with a large SacI-SalI fragment of Chimera G (4493 nucleotides).

To make chimera GD, the SacI-SalI fragment (725 nucleotides) of Chimera D was ligated with a large SacI-SalI fragment of Chimera H (4493 nucleotides). To make chimera HC, the SacI-SalI fragment (725 nucleotides) of Chimera C was ligated with a large SacI-SalI fragment of Chimera H (4514 nucleotides). Chimera HD was made by ligating a SacI-SalI fragment (725 nucleotides) of Chimera D with a large SacI-SalI fragment of Chimera H (4514 nucleotides). Chimera I was constructed by ligating a BamHI-Bsu36I fragment (1324 nucleotides) of Chimera GD with a large Bsu36I-BamHI fragment of MN-TCLA (3894 nucleotides). To make chimera J, a Bsu36I-SalI fragment (1175 nucleotides) of Chimera B was ligated with a large SalI-Bsu36I fragment of MN-TCLA (4043 nucleotides). To make chimera K, a SacI-SalI fragment (725 nucleotides) of Chimera C was ligated with a large SalI-SacI fragment of Chimera I (4493 nucleotides). To make chimera L, a SacI-SalI fragment (725 nucleotides) of Chimera C was ligated with a large SalI-SalI fragment of Chimera J (4493 nucleotides). Chimera M was constructed by ligating a SacI-SalI fragment (725 nucleotides) of Chimera D with a large SalI-SacI fragment of Chimera I (4493 nucleotides). To make chimera N, a SacI-SalI fragment (725 nucleotides) of Chimera D was ligated with a large SalI-SacI fragment of Chimera J (4493 nucleotides).

The following eight chimeras were constructed to study possible gp120-gp41 interactions. To construct chimera O, a BamHI-DraIII fragment (592 nucleotides) of MN-P was ligated with a large DraIII-BamHI fragment of Chimera C (4626 nucleotides). To make chimera P, Chimera H was first mutated to introduce EcoRV site at nucleotide 636 forming Chimera H (+EcoRV). The DraIII-EcoRV fragment (268 nucleotides) of Chimera H (+EcoRV) was then ligated with a large EcoRV-DraIII fragment of Chimera C (4950). To make chimera Q, the EcoRV-SacI fragment (914 nucleotides) of Chimera H (+EcoRV) was ligated with a large SacI-EcoRV fragment of Chimera C (4304 nucleotides). This clone was called Qa. The EcoRV site on Qa was then deleted to complete construction of Chimera Q. To make chimera R, Chimera Q was first mutagenyzed to introduce an EcoRV site at nucleotide 636. The BamHI-EcoRV fragment (636 nucleotides) of Chimera O (+Eco RV) was ligated with a large BamHI-EcoRV fragment of Chimera Q (4577 nucleotides).

Finally, the EcoRV site was mutagenyzed back to the Chimera Q sequence at nucleotide 636 to complete construction of Chimera R. Chimera S was constructed by first mutating Chimera C to introduce the four mutations, 300K/N, 345R/S, 398P/S and 418N/S. Then, the BglII-BglII fragment (571 nucleotides) of Chimera C containing the four mutations was ligated with a large BglII-BglII fragment of chimera R (4642 nucleotides). The same BglII-BglII fragment (571 nucleotides) of chimera C containing the four mutations was also ligated with a large BglII-BglII fragment of MN-TCLA (4642 nucleotides) to make chimera T. To make chimera U, the BamHI-DraIII fragment (368 nucleotides) of MN-P was ligated with a large BamHI-DraIII fragment of MN-TCLA (4845 nucleotides). To make chimera V, the BamHI-SacI fragment (1550 nucleotides) of chimera R was ligated with a large BamHI-SacI fragment of MN-TCLA (3663 nucleotides).

Site-directed mutagenesis procedures were carried out using pfu polymerase (Quick Change Mutagenesis Kit, Stratagene) by following the instructions of the manufacturer. The reactions were performed in an automated thermal cycler (Perkin-Elmer model 2400). Nucleotide sequences were confirmed by sequencing using the ABI PRISM big dye-terminator method (Applied Biosystems 3100 Genetic Analyzer).

Additional mutant envelope genes were constructed to contain specific segments or sequences of each of the parental genes. The construction of Chimera C has been described previously. Chimera P was constructed by replacing the V1/V2 region sequence of Chimera C with the corresponding sequence from MN-P. To prepare for construction of Chimera P an EcoRV site was introduced by mutagenesis into the MN-P sequence at nucleotide 636. The DraIII-EcoRV sequence from the modified MN-P sequence was transferred into the corresponding site of Chimera C. Chimera P/7CD4bs (defined below) was constructed by transferring the EcoRV-SacI fragment from Chimera C/7CD4bs (defined below) into the corresponding site of Chimera P.

Mutagenesis procedures were carried out by using primers designed with single or double nucleotide changes and the Pfu polymerase (Quick Change Mutagenesis Kit from Stratagene) following the instruction of the manufacturer, as described previously (Dong et al. (2003) J. Virol. 77, 3119-3130). The reactions were performed in an automated thermal cycler (Perkin-Elmer model 2400). Nucleotide sequences of each mutation introduced were confirmed by sequencing by using the ABI PRISM dye-terminator method (Perkin-Elmer).

### Example 3

### Pseudovirus Construction and Assays for Infectivity and Neutralization

Pseudoviruses expressing envelope glycoproteins derived from various env plasmids were constructed by using pSV7d-env and pNL4-3.Luc.E-R- plasmids as described previously (Leavitt et al. (2003) J. Virol. 77, 560-570; Park & Quinnan (1999) J. Virol. 73, 5707-5713; Park et al. (1998) J. Virol. 72, 7099-7107; Park et al. (2000) J. Virol. 74, 4183-4191). Briefly, the env plasmid and pNL4-3.Luc.E-R-DNA were cotransfected into 30% confluent 293T cell cultures by the calcium phosphate method (Promega). The culture medium was replaced with fresh medium containing 1 μM sodium butyrate at 18-24 hours post-transfection. At 44-48 hours after transfection, the pseudovirus-containing supernatants were harvested, filtered through a 45 pm-pore-size sterile filter (Millipore), supplemented with additional fetal bovine serum to a final concentration of 20% and stored at −80° C. if not used immediately. To measure the infectivity of pseudoviruses, a luminescence assay with HOS-CD4-CXCR4 cells was used as previously described. HOS-CD4-CXCR4 cells (1×10⁴ cells/1 ml) were inoculated with a serially diluted pseudovirus in 96-well plates with flat-bottom wells. The cultures were incubated for three days at 37° C. with 5% carbon dioxide, after which the cells were washed with 150 μl phosphate-buffered saline (pH 7.4) and lysed with 15 μl of cell lysis buffer (Promega) for thirty minutes. The amount of luciferase activity in each well was determined with 50 μl of substrate (Promega) in an EG&G Berthold MicroLumat Plus luminometer (Wallac).

The neutralization phenotype of each pseudovirus was tested in a manner similar to that of the infectivity assay, except that aliquots of serially diluted serum or antibody were mixed with appropriately diluted pseudovirus and incubated for 1 h at 4° C., after which HOS-CD4-CXCR4 cell suspensions were added and incubated for three days at 37° C. with 5% carbon dioxide.

### Example 4

### gp120 Dissociation Assay and ELISA

Spontaneous and ligand-induced gp120 dissociation was assessed by enzyme-linked immunosorbent assay (ELISA). Briefly, pseudotyped viruses in transfected cell culture supernatants were filtered, sedimented by centrifugation at 21,130×g for 2 h at 4° C. (Tomy Tech USA), washed once with prechilled PBS by centrifugation and resuspended in PBS with 10% FBS in one-fortieth of the initial volume. Similar results are obtained when pseudotyped viruses are sedimented as pellets or onto a sucrose cushion then into pellets (Park et al. (2000) J. Virol. 74, 4183-4191; Park et al. (1999) J. Virol. 73, 5707-5713).

Each aliquot of concentrated pseudotyped virus was incubated at 37° C. for one hour with 5 μg/ml of sCD4 or PBS. The pseudotyped particles were then separated from dissociated gp120 by centrifugation at 21,130×g for two hours. The level of gp120 dissociation was determined by comparing gp120 antigen in the samples of the supernatants and pellets measured by ELISA. The amount of p24 antigen in both supernatant and pellet samples were also measured. The ELISA assay was conducted by antigen capture, as described previously (Park et al. (2000) J. Virol. 74, 4183-4191; Park et al. (1999) J. Virol. 73, 5707-5713). Briefly, each well of the Immulon-2 microtiter plate (Dinex Technology) was coated with a human anti-HIV-1 IgG. The antigen was prepared in lysis buffer and diluted in blocking reagent was applied, and bound antigen was detected using either sheep anti-gp120 or rabbit anti-p24 antibody. Bound detection antibodies were assayed using biotinylated anti-sheep (Vector Laboratories) or anti-rabbit antibody, followed by avidin-conjugated horseradish peroxidase (Vector Laboratories) and then orthophenylenediamine (Abbot Diagnostics Laboratories) or TMB (Kirkegaard and Perry Laboratories) substrate development, respectively. Standard antigen controls used in the assays consisted of serial dilutions of p24 and MN strain gp120, each obtained form the NIH-ARRRP (ARRRP 382 and 3927, respectively).

### Example 5

### Location of gp120 Core Mutation in the gp120 Atomic Structure

The PDF file used is from Protein Data Bank 1GC1 (Kwong et al. (1998) Nature 393, 630-631 which is herein incorporated by reference in its entirety). Figures represent the location of the mutation was drawn with PCMolecule2 (version 2.0.0 Molecular Ventures, Inc.) and with Corel Draw (version 8.369).

### Example 6

### Nucleotide and Amino Acid Sequences of Primary MN Clones

Two clones derived from the primary MN virus pool were selected based on being functional in infectivity assays when pseudotyped on virus particles. These clones are designated MN-P and MN-P14. The nucleotide sequences of these env genes were determined, and the predicted amino acid sequences were compared to those of the MN-TCLA and MN-E6 clones, described previously, as shown in FIG. 1 (Park et al. (2000) J. Virol. 74, 4183-4191; Park et al. (1999) J. Virol. 73, 5707-5713). The MN-TCLA and MN-E6 clones were 98% similar to each other, but only 91.5-92.5% similar to the MN-P and MN-P14 clones. The MN-P and MN-P14 clones were 95.6% similar to each other. Among 88 amino acid residues at which the MN-P or MN-P14 clones differed from the MN-TCLA clone, the MN-P and MN-P14 clones both varied at 64 residues. Additionally, both the MN-P and MN-P14 clones had unusual insertional mutations in variable region 1 (V1) that resulted in two extra cysteine residues with probable formation of an extra disulfide bond. In both cases, it appeared that this insertion mutation had resulted from a duplication mutation. Thus, the MN-P clone was similar to the MN-P14 clone, and is reasonably likely to be representative in the same sense of other clones that were present in the virus quasispecies mixture from which they were derived.

Polymorphisms found comparing highly neutralization resistant MN-P and neutralization sensitive MN-TCLA clones in the gp120 include three in the C1 region, seven in the V1/V2 region, seven in the C2 region, four in the V3 region, three in the C3 region, two in the V4 region, two in the C4 region, and two in the V5 region of gp120. The polymorphisms in gp41 included seven in the amino terminal segment of gp41 proximal to the disulfide-bonded loop, most of this region is alpha helical in structure in the fusion active state and the region will be subsequently referred in this paper as the leucine zipper domain (LZ), seven in the membrane proximal alpha helical region (AH), one in the transmembrane (TM) and nine in the cytoplasmic tail (CT).

The MN-P clone differed from MN-TCLA at five of the six residues at which mutations have been previously reported as causing the differences in neutralization resistance phenotypes of MN-E6 and MN-TCLA (Park et al. (2000) J. Virol. 74, 4183-4191). At three of these residues the mutations that distinguished MN-P and MN-E6 from MN-TCLA were the same, V420L, N564H, and Q582L. At two residues the mutation that distinguished MN-P and MN-E6 from MN-TCLA were different, 1460E/N and L544P/Q. Thus, MN-E6 may have been derived from a clone in a quasispecies mixture that was a common ancestor to the MN-P clone and had persisted in the MN-TCLA virus pool.

### Example 7

### Neutralization Resistance and High Infectivity Phenotypes of MN-P

The neutralization sensitivity and infectivity of the MN-P and MN-TCLA clones and various chimeric genes derived from them are presented in FIG. 2. The infectivity and neutralization results shown are the mean results of eight comparative tests of the MN-TCLA and MN-P clones. The results shown for each of the other clones shown are the mean result for three to five tests per clone; each one of these tests was included in one of the experiments shown comparing the MN-TCLA and MN-P clones. In addition, each of the clones regarding which direct comparisons are made in this section were included in repeated experiments in which they were compared directly. The MN-P clone was 1250-fold more infectious in HOS-CD4-CCR5 cells than the MN-TCLA clone, and 256-fold more resistant to neutralization by the reference serum HNS2. The neutralization resistance and infectivity of MN-P is similar to those characteristics of other primary HIV-1 envelopes that have been tested (Zhang et al. (2002) J. Virol. 76, 644-655; Zhang et al. (1999) J. Virol. 73, 5225-5230).

The chimeric clones were constructed to permit evaluation of regions of the MN-P gene responsible for the high infectivity and neutralization resistance phenotypes of MN-P. Chimera A derived its 5′ sequences, up to the SacI site, located four codons downstream of the coding sequence for the gp120-gp41 cleavage site, from MN-TCLA and its 3′ sequences from MN-P. It was consistently intermediate in infectivity and neutralization resistance in comparison to MN-P and MN-TCLA. Chimera B derived its 5′ sequences, up to the SacI site, from MN-P and its 3′ sequences from MN-TCLA. It was less infectious and resistant to neutralization than chimera A, but slightly more infectious and neutralization resistant than MN-TCLA. These results indicate that sequences in both gp120 and gp41 contribute to the high infectivity, neutralization resistance phenotype of MN-P.

Previous studies demonstrated that the high infectivity, neutralization resistance phenotype of the MN-E6 clone was attributable to functional interactions between the carboxy-terminal region of gp120 and the LZ region of gp41. Chimeras C and F were constructed to permit testing of the importance of the LZ region of MN-P. Chimera C, which was constructed by the introduction of the LZ region of MN-P into MN-TCLA, had slightly increased neutralization resistance and infectivity compared to MN-TCLA. Conversely, chimera F, which consisted of mostly MN-P sequences with the LZ region derived from MN-TCLA, was also only slightly more infectious and neutralization resistant than MN-TCLA. These results demonstrated that the high infectivity, neutralization resistance phenotype of MN-P was dependent upon the LZ sequence, but this sequence was not sufficient to impart the phenotype.

The functional interaction of the amino terminus of gp120 with LZ sequences was evaluated by comparison of Chimeras C, F, HC and HD. Chimera HC incorporated sequences from the amino terminus of MN-P gp120 and the amino terminus and cytoplasmic tail of MN-P gp41 into the MN-TCLA background. Chimera HC was substantially more infectious and neutralization resistant than Chimeras C or F. It is likely that these phenotypic characteristics of chimera HC reflect functional interactions between the amino terminus of gp120 and the LZ region of gp41. The possibility that the cytoplasmic domain of gp41 could contribute to some of the phenotypic effects remains to be determined.

The possibility of functional interaction between the carboxy terminus of gp120 and the LZ region is indicated by comparison of Chimeras C, G, H, and GC. There were relatively small differences between Chimera G and MN-TCLA, while chimera GC was substantially more infectious and neutralization resistant. These comparisons indicate that the relatively high infectivity, neutralization resistance phenotype of Chimera GC is due to functional interactions between the carboxy terminus of gp120 and the LZ. Results of testing of chimeras I, J, K, L, M, and N further support the interpretation that functional interactions occur between different regions of the carboxy terminus of MN-P gp120 and the LZ contributing to the neutralization resistance, high infectivity phenotype.

Comparisons of Chimeras A, C, D, GD, E, and HD indicated a functional interaction of the AH region of gp41 with the LZ region. Chimera A contains sequences of the entire MN-P gp41, and was substantially more infectious and neutralization resistant than either chimera C or D. Chimera D contained the MN-P sequences encoding the AH of the gp41 ectodomain. Conversely, chimera E had MN-P sequences throughout, except for the AH region, and it was significantly less infectious than MN-P. Chimeras GD and HD combined MN-P AH sequences with sequences from other regions of MN-P, excluding the LZ region, and no complementation was observed. These results indicate a specific functional interaction between the LZ and carboxy-terminal regions of gp41 contributing to the neutralization resistance, high infectivity phenotype of MN-P.

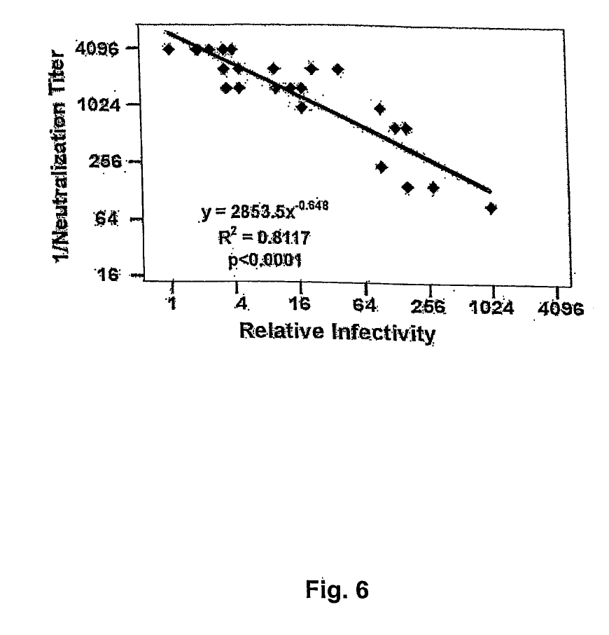

Based on the analyses presented here, the results presented here demonstrate functional interactions of the MN-P LZ region with the amino and carboxy termini of gp120 and the carboxy-terminal region of gp41. These results indicate that the LZ region plays a significant role in organizing the functions of the HIV-1 envelope protein complex. Moreover, there was a general correspondence between effects of specific mutations on the two characteristics of the phenotype being evaluated. To test the possibility that these multiple functional interactions between the LZ and other regions of the envelope proteins were modulating both characteristics simultaneously by common mechanisms, we tested whether there was a statistical correlation between the characteristics, as shown in FIG. 6. A strong, statistically significant correlation was obtained.

### Example 8

### Localization of MN-P Mutations on the Core of the Atomic Structure of gp120

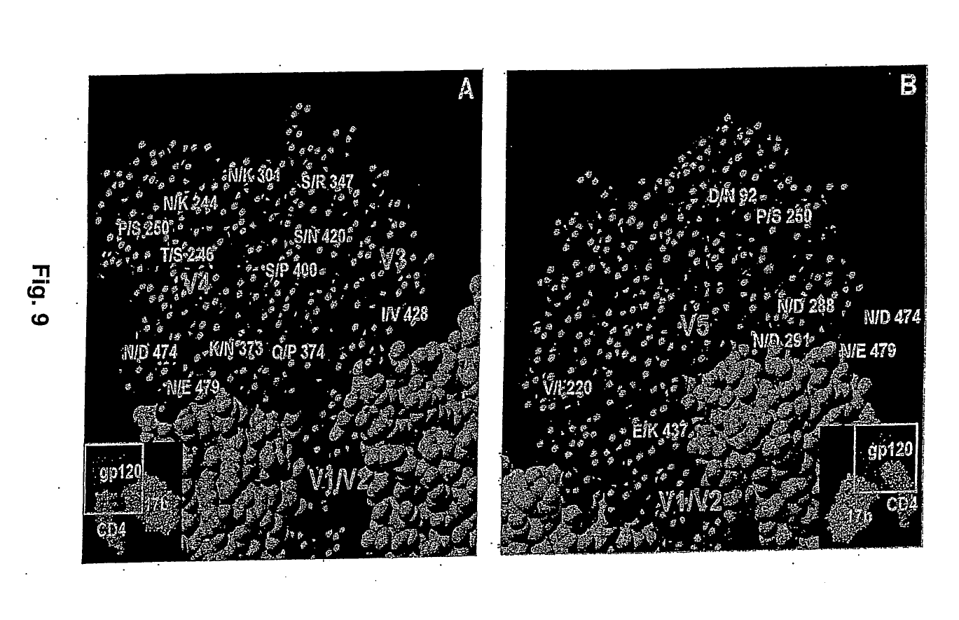

The localization of mutations in the core structure of MN-P gp120 was examined, as illustrated in FIG. 9. The mutated residues are identified according to the numbering system of Kwong et al. (1998) Nature 393, 630-631. Only those mutations affecting residues visualized in the gp120 core structure are shown. Mutations in the extreme amino terminus first ninety amino acids and the V1/V2 and V3 regions of gp120 are not shown in the Figure. Seven of the mutations were identified as being localized in or around the rim of the CD4 binding pocket. These seven mutations are shown as Asp287, Asp290, Asn371, Pro372, Lys435, Glu466, and Asp472. Two of the mutations are localized in the region of gp120 considered to be the co-receptor binding domain, and are identified as Ile219 and Val426. Four of the mutations are localized to the pole of gp120 described as the inner domain, including Asn91, Lys243, Ser245 and Ser249. Four mutations were localized in the outer domain of gp120, but were distant from the CD4 or co-receptor binding sites. These four mutations were Lys300, Arg345, Pro398, and Asn418. The distributions of these mutations indicates that some or all of them functioned in aggregate to enhance the infectivity and neutralization resistance of MN-P by modulating the interactions of gp120 with its ligands, including CD4, co-receptor and gp41.

### Example 9

### Specific Mutations Distant from Binding Sites Contribute to High Infectivity Phenotype of MN-P

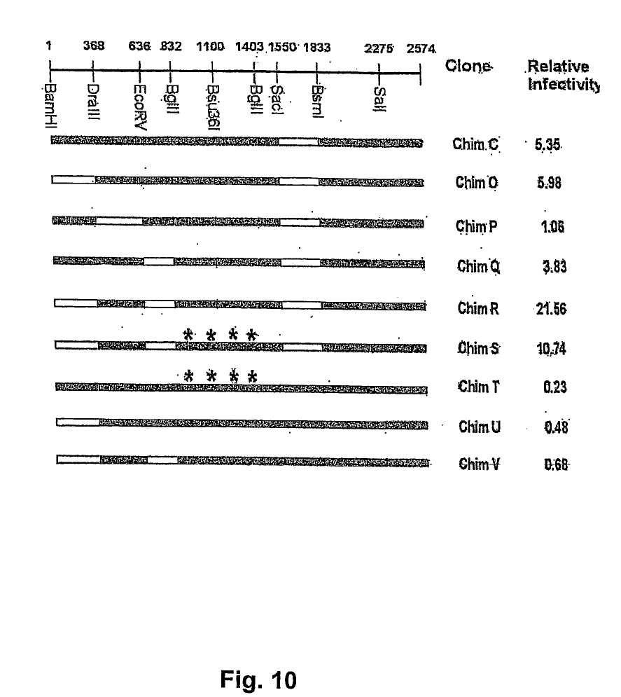

Chimeric envelopes were constructed to evaluate the specific contributions of gp120 amino terminal sequences, including V1/V2 sequences, to the neutralization resistance, high infectivity phenotype. V1/V2 sequences are included in the DraIII 368-EcoRV 636 segment shown in FIG. 10. The potential contribution of the V1/V2 region mutations to phenotype was evaluated by comparison of Chimeras C, HC, O, P, and Q (FIGS. 2 and 6). Infectivity of clones O, P, and Q was similar to that of chimera C, indicating that sequences in more than one subsegment of the BamHI-BglII (832 nucleotides) segment are required to determine the phenotype of chimera HC.

Chimera R was constructed for further study of the role of gp120 core structure mutations in the amino terminus in determining the infectivity phenotype. Chimera R includes all of the mutations in the BamHI-BglII segment of MN-P, except those in the V1/V2 region. While chimera R was somewhat less infectious than chimera HC (FIG. 2), it was significantly more infectious than chimeras C, O, or Q, indicating that the two segments in the amino terminal region of gp120 functioned together, in the context of MN-PLZ sequences, to determine enhanced infectivity. Chimera S was constructed by introducing the outer domain core structure mutations not associated spatially with the CD4 or co-receptor binding sites into chimera R. Chimera S was less infectious than chimera R indicating that the effect of the mutations in the non-V1/V2 segments of the amino terminus of MN-P gp120 on infectivity was not further enhanced by these outer domain mutations. Results shown from testing of chimeras T, U, and V further support the interpretations presented in herein.

### Example 10

### gp120-gp41 Dissociation

Non-covalent bonding between residues of gp120 and gp41 maintains the association between the two molecules in the functional envelope protein complex. Because of the possibility that mutations in gp120 modulated the interaction between gp120 and gp41 in a way that contributed to the high infectivity phenotype, the effects of MN-P mutations on the stability of the gp120-gp41 association were tested. Furthermore, since binding of gp120 to CD4 affects its association with gp41 in some cases, and the concerted interactions between gp120 and its ligands may determine its infectivity phenotypes, the effect of sCD4 binding on gp120-gp41 dissociation was tested. To measure the dissociation of gp120 from gp41, ELISA was used to determine the separation of particle-free and particle-associated gp120 that resulted from centrifugation of pseudotyped virus particles. It has been previously found that this technique of separating virus particles from media supernatants by centrifugation of the particles into pellets yields comparable results to those obtained when particles are collected on sucrose cushions.

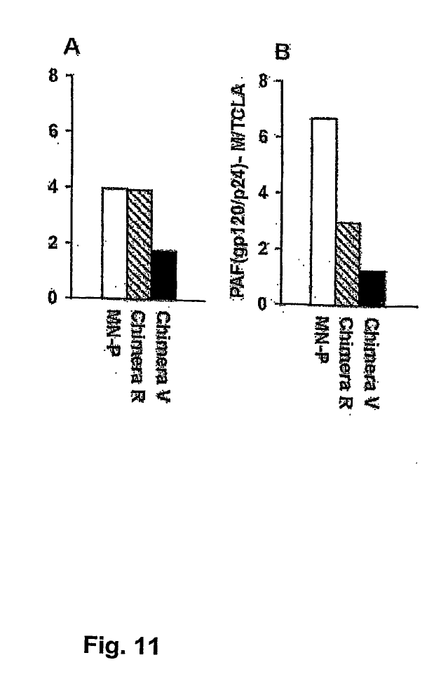

Experiments were conducted comparing the spontaneous and sCD4-induced dissociation of gp120 from gp41 for MN-TCLA, MN-P, and chimeras R and V. Chimera R contains all of the MN-P mutations localized to the inner domain of gp120 on the atomic structure of the molecule, as well as two mutations in the amino terminus of the MN-P, A64V and E84Q. It also contains the MN-P LZ sequences. Chimera V contains the same gp120 MN-P sequences, but contains the MN-TCLA LZ sequences. The results of experiments testing the dissociation of gp120 from these pseudotyped viruses are summarized in Table 1 and FIG. 11. The effectiveness of separation of particles from medium components was evaluated by determining the relative amounts of p24 in pellets and supernatants. The percentage of p24 in the supernatants averaged between 14.9% (MN-P plus sCD4) and 28.8% (chimera V plus sCD4). In each case, these proportions were similar in the presence and absence of sCD4.

TABLE 1
Comparative sedimentation analysis of particle association of gp120 and p24 of MN-
TCLA, MN-P and chimeric HIV-1 env genes.

| | | MN-TCLA | | MN-P | | Chimera R | | Chimera V | |
|---|---|---|---|---|---|---|---|---|---|
| | | gp120^{a} | p24^{a} | gp120^{a} | p24^{a} | gp120^{a} | p24^{a} | gp120^{a} | p24^{a} |
| Ligand | | ng/ml ± sem | μg/ml ± sem | ng/ml ± sem | μg/ml ± sem | ng/ml ± sem | μg/ml ± sem | ng/ml ± sem | μg/ml ± sem |
| None | P | 6.4 ± 2.7 | 11.5 ± 2.6 | 25.3 ± 6.1 | 6.7 ± 2.4 | 25.2 ± 3.3 | 15.2 ± 2.8 | 11.2 ± 4.8 | 15.8 ± 2.2 |
| | SN | 2.9 ± 0.8 | 2.7 ± 1.3 | 2.9 ± 1.8 | 1.4 ± 0.6 | 6.2 ± 2.1 | 3.5 ± 1.8 | 5.2 ± 1.6 | 5.5 ± 1.4 |
| sCD4 | P | 6.4 ± 3.0 | 12 ± 2.2 | 25.6 ± 5.8 | 6.6 ± 2.2 | 20.1 ± 3.4 | 15.4 ± 2.1 | 11.6 ± 4.3 | 15.3 ± 2.1 |
| | SN | 2.9 ± 1.0 | 2.6 ± 1.2 | 14.0 ± 4.2^{b} | 1.1 ± 0.5 | 15.0 ± 5.7 | 4.4 ± 1.9 | 5.5 ± 1.8 | 6.1 ± 2.0 |

^{a}Amount of gp120 and p24 in pellets (P) and supernatants (SN) of centrifuged suspensions of viruses pseudotyped with each envelope with or without pre-exposure to sCD4. Centrifugation was for two hours at 4° C., 21130 × g. Viruses were pre-incubated with sCD4 at 5 μg/ml for one hour at 37° C. Means (±SEM) of gp120 and p24 antigen concentrations determined by ELISA of MN-TCLA(n = 5), MN-P (n = 5), Chimera R (n = 3) and Chimera V (n = 3). All tests shown included MN-P for comparison to other clones.
^{b}Mean value differs from that in the samples not preincubated with sCD4 at P < 0.05; Non parametric, Wilcoxon Signed Ranks Test.

The amount of gp120 measured in association with virus particles was consistently greater for MN-P than MN-TCLA (Table 1 and FIG. 6). This difference averaged approximately four-fold. The difference was greater, 6.7-fold, when the amount of gp120 in the pellets was expressed in proportion to the amount of p24. The amount of gp120 associated with chimera R pellets was also greater than with MN-TCLA pellets by 3.9-fold, and this difference remained at 3-fold when expressed as proportional to p24. There was slightly more gp120 associated with chimera V than MN-TCLA particles, by 1.75-fold, but this difference was only 1.3-fold when expressed in proportion to p24. Thus, chimera R resembled MN-P in the greater association of gp120 with viral particles, while chimera S was very similar to MN-TCLA.

Spontaneous dissociation of gp120 from MN-TCLA, in the absence of sCD4, was 31.2%, significantly greater than from MN-P, which was 10.3%. Spontaneous gp120 dissociation of gp120 from chimera R was lower than from MN-TCLA, 19.8%, while dissociation from chimera V was nearly identical to MN-TCLA, 31.8%. When bound by sCD4, there was no change in the release of gp120 from MN-TCLA, but gp120 release from MN-P increased more than 3-fold to 35.3%. Binding by sCD4 significantly enhanced gp120 release from chimera R to 42.7%, but had no significant effect on release from chimera V. Thus, chimera R also resembled MN-P with respect to spontaneous and sCD4-induced release of gp120 from virions, while chimera V closely resembled MN-TCLA in these respects.

### Example 11

### Regions of the MN-P Gene Contributing to Resistance to Neutralization by sCD4

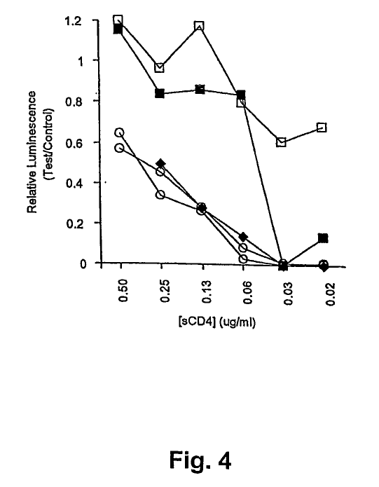

Neutralization resistance, high infectivity phenotype of the MN-P clone is dependent upon functional interactions between sequences from the region of the MN-P gene encoding the gp41 HR1 with sequences in other regions of gp120 and gp41. FIG. 3 illustrates the structure of chimeric genes C, F, A, GC, and HC, which we used in that previous study, as well as the neutralization titers reported therein using an HIV-1 immune human serum and viruses pseudotyped with the respective envelopes. FIG. 3 also illustrates the structures of additional chimeric genes which were used in the present study to analyze intramolecular interactions that determine resistance to neutralization by sCD4. Chimera C has MN-P HR1 region sequences in the MN-TCLA backbone. Chimera F is the reciprocal construct, with the MN-TCLA HR1 region in the MN-P backbone. The amino terminus of gp120, the carboxy terminus of gp120, or the carboxy terminus of gp41 from MN-P were each introduced into Chimera C to form Chimeras HC, GC, and A, respectively. These three chimeras were used to evaluate whether functional interactions between different regions of gp120 or gp41 and the HR1 region contributed to resistance to neutralization by sCD4. The reasons for construction of Chimeras P and R, shown at the bottom of FIG. 10 and discussed below. As shown in FIG. 4, Chimera GC was significantly more resistant to neutralization by sCD4 than Chimera C, but less resistant than MN-P. Chimeras A and HC were similar to Chimera C. These results indicate that functional interactions between the carboxy terminal half of gp120 and the HR1 contributed to resistance to neutralization by sCD4, but that interactions not reflected in the chimeric genes included in these comparison also contributed to the resistance.

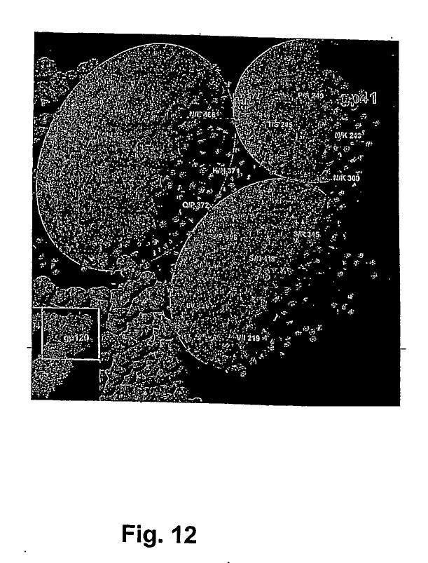

Amino acid substitutions in the segment of gp120 contributed by MN-P to Chimera GC. The distribution of mutations distinguishing MN-P from MN-TCLA throughout the linear sequences of the genes and on the core structure of gp120 is illustrated in FIGS. 9 and 12. There were four distinguishing amino acid substitutions in V3 and twelve additional substitutions in the segment of MN-P gp120 included in Chimera GC (amino acids 284 to 474 of MN-P). Seven of the twelve non-V3 substitutions were seen on the model of the atomic structure (FIG. 9) to be in or near the CD4 binding pocket of gp120. Two of these, N/D290 and E/K435, involved amino acids that aligned with residues demonstrated by Kwong et al. (1998) Nature 393, 648-659 to form direct contacts with CD4. Two other mutations, KQ/NP371-2 and N/E466, affected residues immediately adjacent to residues that form direct contacts with CD4. In addition, six of the seven substitutions involved charge alterations (D/N287, N/D290, K/N371, E/K435, N/E466, and N/D472), and three involved gain or loss of potential N-linked glycosylation sites (D/N288, K/N371, and N/E466). Of the remaining five gp120 core substitutions in the segment from amino acids 284-474, but not within or in close proximity to the CD4 binding domain, one is believed to be located in the center of the co-receptor binding site (I/V426). This mutation was seen previously in the MN-E6 clone, and did not, by itself, confer resistance to neutralization by sCD4 in that context. The remaining four mutations in this segment are located on the surface of the gp120 outer domain, distant from the CD4 and co-receptor binding sites. Based on these considerations, mutations in or near the CD4 binding pocket altered sensitivity to neutralization by sCD4 through substitutions at residues that directly bond CD4 or at adjacent residues that modify their interactions with CD4, such as through, charge alterations, or by alteration of glycosylation sites that may have steric effects on CD4 binding.

### Example 12

### Effects of Mutations in and Near the CD4 Binding Site on Resistance to Neutralization by sCD4

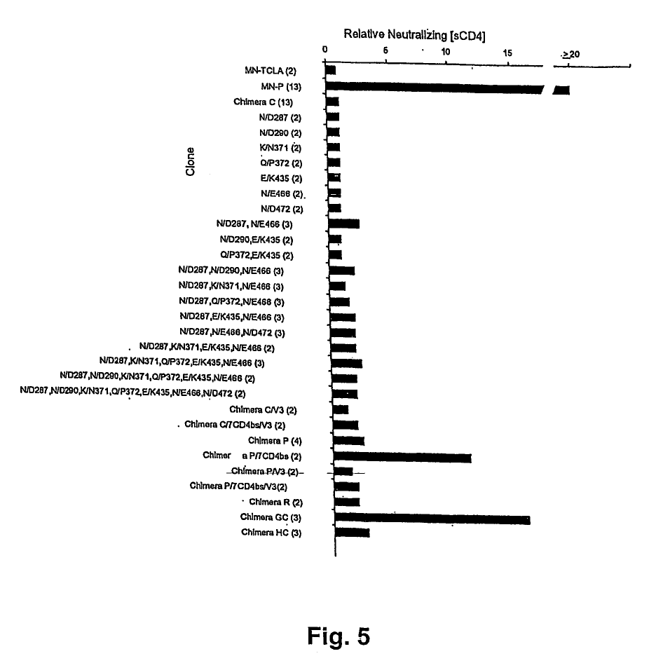

To test the significance of mutations in or near the CD4 binding domain, we pursued a strategy using Chimera C as a platform for testing the effect of other mutations on resistance to neutralization by sCD4. The comparative sensitivity to neutralization by sCD4 of MN-TCLA, MN-P, and Chimera C are shown near the top of FIG. 5. The MN-P clone is more than 50-fold more resistant to sCD4 neutralization than the MN-TCLA clone. In most of the assays represented in FIG. 5, the maximum concentration of sCD4 used was 1.0 μg/ml, so that neutralization of MN-P was not achieved. Based on the assays reported here we calculated that the MN-P clone was at least twenty-fold more resistant than MN-TCLA. Chimera C was not significantly more resistant to neutralization by sCD4 than MN-TCLA. The seven mutations located in and near the CD4 binding site that distinguished MN-P from MN-TCLA were introduced into Chimera C, singly and in various combinations, as shown in FIG. 5. Only small differences between Chimera C and any of these mutants were noted, including the clone of Chimera C containing all seven of the mutations (this clone is referred to subsequently as Chimera C/7CD4bs). These results indicated that the resistance of MN-P to neutralization by sCD4 was not due primarily to changes in the CD4 binding site.

The possible importance of mutations represented in Chimera R with respect to resistance to neutralization by sCD4 was next considered. Chimera R was of interest because of previous studies that demonstrated that mutations in the amino terminus of gp120 determine a functional interaction with HR1 sequences that confers a sCD4-response phenotype. MN-TCLA has a high spontaneous gp120-gp41 dissociation phenotype, but does not display enhanced dissociation consequent to sCD4 binding. In contrast, MN-P has a low rate of spontaneous gp120-gp41 dissociation, which is increased substantially by sCD4 binding. Chimera R has a gp120-gp41 dissociation phenotype like MN-P. The mutations that distinguish Chimera R from Chimera C include the four mutations that can be seen to cluster in the inner domain in FIG. 3 (D/N91, N/K243, T/S245, and P/S249), as well as two that are in the extreme amino terminus of gp120 and not visualized in the crystallographically-determined gp120 core structure (V/A64 and Q/E84). We have previously suggested, based on their effects on CD4-induced dissociation of gp120 from gp41, that these six residues contribute to a potential gp41-binding site on gp120. Remarkably, even though these mutations determine a sCD4-response phenotype, they did not significantly affect sensitivity to neutralization by sCD4 (FIGS. 3 and 5).

The contribution of sequences in the V1/V2 or V3 regions to sCD4 resistance was considered next. Previous reports have demonstrated that variable regions 1 and 2 may partially mask access to the CD4 binding site (Rizzuto et al. (1998) Science 280, 1949-1953, 44). The MN-P clone has an unusual duplication in V1, as well as a number of other mutations that distinguish it from the V1/V2 region of MN-TCLA. To permit testing of the role of the V1/V2 region in sCD4 resistance, we constructed Chimera P, which consisted of MN-TCLA sequences throughout most of the gene, except for the V1/V2 and HR1 regions that were derived from MN-P, as illustrated in FIG. 3. Additional genes were constructed using the Chimera P backbone, by introduction of the segment containing the seven mutations in or near the CD4 binding site (Chimera P/7CD4bs), four mutations (YN/NY311-12, R/K313, and T/K315) that distinguish the proximal limb of MN-P V3 region from that of MN-TCLA (Chimera PN3), or both of these sets of mutations (Chimera P/7CD4bs/V3). Chimera P was only 1.9-fold more resistant to neutralization by sCD4 than Chimera C (FIG. 5), whereas Chimera P/7CD4bs was 11.3-fold more resistant. The mean 50% neutralizing concentrations of sCD4 for Chimera C, Chimera P, and Chimera P/7CD4bs, respectively, were 16, 31, and 176 ng/ml. Unexpectedly, addition of V3 region mutations to Chimera P/7CD4bs, forming Chimera P/7CD4bs/V3, abrogated the neutralization resistance. These results indicate a functional relationship between residues near the CD4 binding domain and the V1/V2 region that contributed to the sCD4 resistance of MN-P, as well as a further functional relationship between these regions and V3.

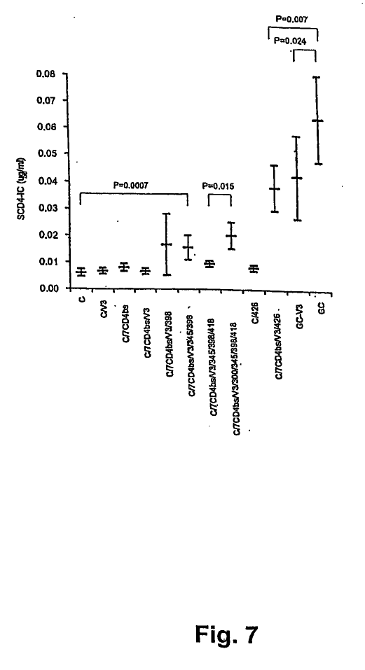

We next considered the structure of Chimera GC, which is relatively resistant to neutralization by sCD4. Chimera GC incorporates carboxy terminal sequences from MN-P gp120 into Chimera C. The gp120 sequences from MN-P in Chimera GC include the seven CD4 binding domain mutations discussed above, the V3 region mutations, one mutation thought to be located in the center of the co-receptor-binding site (I/V426), and four mutations localized in a loose cluster in the outer domain of gp120 (N/K300, S/R345, S/P398, and S/N418). As shown in FIG. 5, lower part, there were only minor differences noted in sCD4 resistance among Chimeras C, C/V3, C/7CD4bs/V3, P, P/V3, and P/7CD4bs/V3. Therefore, the interactions between residues in V3 and other residues that are mutated in Chimera GC contributed to neutralization resistance. The S/P398, S/R345, S/N418, and N/K300 outer domain mutations were introduced sequentially, and studied for effect, as shown in FIG. 7. Among these, both the S/P398 and N/K300 mutations appeared to contribute small, but statistically significant effects. The co-receptor binding site mutation, I/V426 had no effect on sCD4 neutralization when introduced into Chimera C, but had as significant effect when introduced into Chimera C/7CD4bs/V3. This mutant, Chimera C/7CD4bs/V3/426, was significantly less resistant than Chimera GC, indicating that one or more of the four outer domain mutations were required for expression of the full phenotype of Chimera GC. Finally, the Chimera GC V3 sequences were back-mutated to the MN-TCLA V3 sequence, forming Chimera GC-V3. This clone was also less resistant to neutralization than Chimera GC, indicating that V3 sequences were required for the full neutralization resistance phenotype. These results demonstrate, therefore, the occurrence of functional interactions between the V3 region and mutated residues in the outer domain, as well as with residue 426 in the co-receptor binding site.

### Example 13

### Sensitivity of Clones to Neutralization

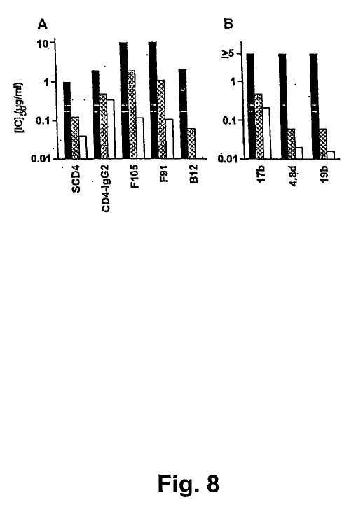

It was of interest to determine if relative sensitivity of clones to neutralization by various CD4 binding domain ligands was consistent. CD4IgG2 tends to be more cross-reactive than sCD4 in neutralization of primary envelopes, but also has greater bulk, which could limit its access to the CD4 binding domain. Among various anti-CD4 binding domain monoclonal antibodies, b12 tends to be more cross-reactive among primary envelopes. Neutralization of viruses pseudotyped with the MN-P, Chimera P/7CD4bs, and Chimera P/7CD4bs/V3 clones by various ligands is shown in FIG. 8. The comparative neutralization of the clones by sCD4 was generally similar to that observed with the monoclonal antibodies against CD4 binding domain epitopes, including b12. MN-P was more resistant to neutralization by each of the ligands than was either of the other two clones. The greater sensitivity to neutralization of Chimera P/7CD4bs/V3 than Chimera P/7CD4bs that was observed with each of the other ligands was not observed with CD4IgG2. These results indicated that the phenomena that determined resistance to neutralization by sCD4 usually acted similarly in determining resistance to neutralization by various CD4 binding domain ligands, and that the mechanism of resistance to neutralization may not have been steric inhibition of ligand binding.

Neutralization by monoclonal antibodies against the co-receptor-binding site (17b and 4.8d) and V3 (19b) is shown in FIG. 8. Fifty percent neutralization of the MN-P pseudotyped virus was not achieved at the highest concentrations tested of these antibodies (2.5 μg/ml). Chimera P/7CD4bs/V3 was more sensitive to neutralization than Chimera P/7CD4bs by 17b (210 versus 480 ng/ml), 4.8d (20 versus 60 ng/ml), and 19b (16 versus 60 ng/ml). These results indicated that the relative sensitivity of these clones to neutralization by sCD4 and monoclonal antibodies against non-CD4 binding domain epitopes was similar. Moreover, the functional interactions between V3 and other regions of gp120 that accounted for the comparative sensitivity of Chimera P/7CD4bs and Chimera P/7CD4bs/V3 also modulated global neutralization sensitivity.

The results of the studies presented here demonstrated that the mechanism of MN-P resistance to sCD4 neutralization, and likely for global neutralization resistance as well, involves multiple functional interactions of the V1/V2 and V3 regions across the surface of the gp120 core structure. The extents of these interactions are illustrated in FIG. 12. Ovals representing the approximate genetic footprints of each of the two loop structures are shown overlaid on the gp120 core. The footprint of the V1/V2 region extends from the stalk of the double loop to the area near the CD4 binding domain, and includes interaction with V3. The footprint of V3 extends from the stalk of the loop, and includes interaction with V1/V2, with the outer domain mutations distant from the binding sites, and with residue 426 in the co-receptor-binding site. These findings indicate that the V1/V2 and V3 loop structures mediate functional interactions at distances among the CD4, co-receptor and gp41 binding sites. Binding of CD4 to the CD4 binding domain induces conformational changes in V1/V2 and gp41 that cause sequential changes in V3 required for co-receptor interaction. The transmission of signals in this manner could well complement changes in the conformation of the gp120 core structure that result from the binding energy changes associated with CD4 interaction. Such an effect might well explain how mutations that are apparently on opposite ends of the complex, such as in the gp41 HR1 and at residue 426 in the co-receptor-binding site, might display very strong functional interactions. The binding of gp120 to each of its ligands results in conformational changes that are transmitted throughout the gp120 core. Interactions with the ligands also results in conformational changes, such that functional signals are transmitted across the surface of the complex. In summary, the primary virus neutralization resistance phenotype evaluated in the present study is the capacity of the HIV-1 envelope complex to undergo various conformational changes, resulting in high efficiency infectivity. This model provides a basis for understanding the nature of epitopes that are important for primary virus neutralization and for designing methods for stabilizing particular conformations of the envelope complex.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto. All references, patents and patent applications referred to in this application are herein incorporated by reference in their entirety.

1-39. (canceled)

40. An isolated HIV-1 envelope protein comprising:
a substitution at a position corresponding to amino acid 91 in gp120;
a substitution at a position corresponding to amino acid 243 in gp120;
a substitution at a position corresponding to amino acid 245 in gp120; and
a substitution at a position corresponding to amino acid 249 in gp120.

41. The HIV-1 envelope protein of claim 40, wherein the substitution at position 91 is D91N.

42. The HIV-1 envelope protein of claim 40, wherein the substitution at position 243 is N243K.

43. The HIV-1 envelope protein of claim 40, wherein the substitution at position 245 is T245S.

44. The HIV-1 envelope protein of claim 40, wherein the substitution at position 249 is P249S.

45. The HIV-1 envelope protein of claim 40, further comprising one or more amino acid substitutions at a position corresponding to an amino acid in gp120 selected from the group consisting of 64, 84, 219, 287, 290, 300, 311, 312, 314, 316, 345, 371, 372, 398, 418, 426, 435, 466, and 472.

46. The HIV-1 envelope protein of claim 40, further comprising one or more amino acid substitutions at a position corresponding to an amino acid in gp120 selected from the group consisting of V64A, Q84E, D287N, D290N, N311Y, Y312N, K314R, K316T, N371K, P372Q, V426I, K435N, E466N and D472N.

47. An isolated HIV-1 protein comprising:
a D91N substitution at a position corresponding to amino acid 91 of SEQ ID NO: 2;
a N243K at a position corresponding to amino acid 243 of SEQ ID NO: 2;
a T245S at a position corresponding to amino acid 245 of SEQ ID NO: 2; and
a P249S a position corresponding to amino acid 249 of SEQ ID NO: 2.

48. The HIV-1 envelope protein of claim 47 further comprising one or more amino acid substitutions at a position corresponding to an amino acid of SEQ ID NO: 2 selected from the group consisting of 64, 84, 219, 287, 290, 300, 311, 312, 314, 316, 345, 371, 372, 398, 418, 426, 435, 466, and 472.

49. A composition comprising the HIV-1 envelope protein of claims 40 or 47.

50. The composition of claim 49 further comprising an adjuvant.

51. The composition of claim 49 further comprising a pharmaceutically acceptable carrier.

52. The composition of claim 49, wherein the composition is formulated for parenteral administration.

53. A fusion protein comprising the HIV-1 envelope protein of claims 40 or 47.

54. The fusion protein of claim 53 further comprising one or more domains of gp41.

55. The fusion protein of 54, wherein the domain of gp41 is selected from the group consisting of leucine zipper, membrane proximal alpha helix, transmembrane segment, and cytoplasmic tail.

56. The fusion protein of claim 54 further comprising one or domains of gp41 having a substitution of one or more amino acids in the domain.

57. The fusion protein of claim 54, wherein the one or more substitutions is in the leucine zipper of gp41.

58. A method of generating protective immune response against HIV-1 in a human comprising administrating an effective amount of the composition of claim 49.
